SEQUENCE LISTING (2 sequences)

SEQ ID NO: 1 - protein, 7 aa, Artificial Sequence
  Description of Artificial Sequence Synthetic peptide
  MOD_RES (1)..(1): 2-aminobenzoic acid
  MOD_RES (7)..(7): Lys(Dnp)
XGIVRAK

SEQ ID NO: 2 - protein, 9 aa, Artificial Sequence
  Description of Artificial Sequence Synthetic peptide
  MOD_RES (9)..(9): Lys(Dnp)
RPPGFSAFK

(19) United States
(12) Patent Application Publication
(10) Pub. No.: US 2024/0181068 A1
(43) Pub. Date: Jun. 6, 2024

(54) SELECTIVE NEUTRAL PH INHIBITOR OF CATHEPSIN B

(71) Applicants: The Regents of the University of California, Oakland, CA (US); The Scripps Research Institute, La Jolla, CA (US)

(72) Inventors: Anthony O'Donoghue, La Jolla, CA (US); Vivian Hook, La Jolla, CA (US); Michael Yoon, La Jolla, CA (US); Dennis Wolan, La Jolla, CA (US)

(21) Appl. No.: 18/546,750

(22) PCT Filed: Feb. 17, 2022
(86) PCT No.: PCT/US22/16798

§ 371 Date: Aug. 16, 2023

Related U.S. Application Data
(60) Provisional application No. 63/150,392, filed on Feb. 17, 2021.

(30) Foreign Application Priority Data
Mar. 19, 2021 (GB) 2103879.9

(51) Int. Cl. A61K 47/64; A61P 43/00

(52) U.S. Cl. CPC A61K 47/64; A61P 43/00

(57) ABSTRACT

Pharmaceutical compositions comprising a peptidic inhibitor of cathepsin B, such as Z-Arg-Lys-AOMK (Z-R-K-AOMK). Methods for inhibiting neutral pH cathepsin B activity, comprising administering to a subject in need an effective amount of a peptidic inhibitor of cathepsin B. Methods for treatment or prevention of a disease comprising administering to a subject in need an effective amount of a peptidic inhibitor of cathepsin B. Methods of producing pH-selective peptide-AMC substrates and novel peptidic-AOMK inhibitors of cathepsin B.

### STATEMENT OF GOVERNMENT SUPPORT

This invention was made with government support under grant Nos. R01NS109075 and T32GM067550 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 24, 2022, is named 24978-0705_SL.txt and is 962 bytes in size.

### TECHNICAL FIELD

The present invention relates to inhibitors of cathepsin B for disease treatment.

### BACKGROUND

Cathepsin B functions in lysosomes for protein degradation and maintenance of cellular homeostasis (1-3). Cathepsin B is a member of the family of cysteine cathepsin proteases that participate in lysosomal protein degradation, together with aspartyl and serine cathepsins (4, 5). Cathepsin B normally functions within the acidic pH 4.6 environment in lysosomes (6-8). However, significant cathepsin B activity occurs at the neutral pH 7.2 of the cytosol and other cellular compartments as well as extracellular locations (9-11).

Evidence for neutral pH locations of cathepsin B functions (12-15) suggests the hypothesis that differential cleavage properties of this enzyme at neutral compared to acidic pH conditions may provide the basis for design and development of a neutral pH selective inhibitor, which represents the purpose of this study. In numerous disease conditions, cathepsin B functions in the cytosol at neutral pH, rather than in lysosomes. Lysosomal leakage of cathepsin B to the neutral cytosol occurs in numerous brain disorders including Alzheimer's disease (12-17), traumatic brain injury (TBI) (12, 18, 19), and neurodegenerative conditions (20-25), and in autoinflammatory and infectious diseases (26-32). Cytosolic cathepsin B initiates apoptotic cell death (33-36) and activates inflammatory IL-1beta production (37-40). Cathepsin B participates in behavioral deficits, demonstrated by cathepsin B gene knockout and inhibitor studies in animal models of TBI, AD, ischemia, and related disorders (12, 41, 42). This enzyme also functions at the neutral pH of the extracellular environment in cancer (43-47) and rheumatoid arthritis (48), as well as at other neutral pH locations including nuclei (49-51).

The prevalence of cathepsin B functions at neutral pH locations emphasizes the critical importance of this study to gain an understanding its neutral pH properties compared to its normal acidic lysosomal features.

Cathepsin B is active at the neutral pH 7.2 (9, 10) of the cytosol (52, 53). as well as at the acidic pH 4.6 (6-8) within lysosomes (4. 52). The 400-fold difference in proton concentration at pH 4.6 compared to pH 7.2 alters the charge state of cathepsin B (12) and its substrates.

### SUMMARY OF THE INVENTION

The disclosure provides that different pH conditions lead to cathepsin B to possess different substrate cleavage preferences at cytosolic neutral pH compared to lysosomal acidic pH. Based on the distinct cathepsin B cleavage properties at neutral pH compared to acidic pH conditions indicated by MSP-MS data, pH-selective peptide-AMC substrates and novel peptidic-(acyloxy)methyl ketone (AOMK) inhibitors of cathepsin B were designed and evaluated. Notably, Z-Arg-Lys-AOMK (Z-R-K-AOMK) was revealed as a potent and selective inhibitor of neutral pH 7.2 cathepsin B activity. This inhibitor displayed high selectivity for cathepsin B compared to other lysosomal cysteine cathepsins. These results demonstrate that the unique pH-dependent cleavage properties of cathepsin B provide the basis for design of Z-R-K-AOMK as a selective inhibitor of neutral pH cathepsin B activity. These findings suggest that the pathogenic, cytosolic, nuclear or extracellular cathepsin B represents a unique form of the enzyme compared to the normal lysosomal cathepsin B.

In embodiments, the invention provides pharmaceutical compositions comprising a peptidic-AOMK inhibitor of cathepsin B. In embodiments, the peptidic-AOMK inhibitor of cathepsin B is Z-Arg-Lys-AOMK (Z-R-K-AOMK), as others as described herein.

In embodiments, the invention provides methods of inhibiting neutral pH cathepsin B activity, comprising administering to a subject in need an effective amount of a peptidic-AOMK inhibitor of cathepsin B. In embodiments, the peptidic-AOMK inhibitor of cathepsin B is Z-Arg-Lys-AOMK (Z-R-K-AOMK), as others as described herein.

In embodiments, the invention provides methods for treatment or prevention of a disease comprising administering to a subject in need an effective amount of a peptidic-AOMK inhibitor of cathepsin B. In embodiments, the peptidic-AOMK inhibitor of cathepsin B is Z-Arg-Lys-AOMK (Z-R-K-AOMK), and others as described herein.

In embodiments, the disease is due to lysosomal leakage of cathepsin B to the neutral cytosol. In embodiments, the disease is a brain disorder such as Alzheimer's disease, traumatic brain injury (TBI), or neurodegenerative conditions. In embodiments, the disease is autoinflammatory or infectious diseases. In embodiments, the disease is cytosolic cathepsin B initiated apoptotic cell death or activation of inflammatory IL-1beta production. In embodiments, the disease is a behavioral deficit, demonstrated by cathepsin B gene knockout and inhibitor studies in animal models of TBI, AD, ischemia, and related disorders. In embodiments, the disease is cancer or rheumatoid arthritis, or a condition at other neutral pH locations including nuclei, plasma membrane or in the extracellular space.

### BRIEF DESCRIPTION OF THE DRAWINGS

FIGS. 1(a)-1(c) show workflow to analyze cathepsin B substrate cleavage site preferences for design of pH-selective inhibitors. (FIG. 1a) Cathepsin B substrate cleavage properties assessed at pH 7.2 and pH 4.6 by multiplex substrate profiling by mass spectrometry (MSP-MS) analyses. The substrate cleavage profiles of cathepsin B at pH 7.2 and pH 4.6 were assessed by MSP-MS analyses. Cathepsin B was incubated (at RT, for 15 and 60 min) at pH 7.2 and pH 4.6 with the peptide library consisting of 228 14-mer peptides designed to contain all neighbor and near-neighbor amino acid combinations. Peptide cleavage products were identified and quantitated by LC-MS/MS analyses. The frequencies of amino acid residues at the P2 to P2′ positions of the P1-1P1′ cleavage sites were assessed. (FIG. 1b) Design of pH-selective peptide—AMC substrates. Substrates representing the preferred residues at P1 and P2 positions at pH 7.2 and pH 4.6 were utilized for development of pH-selective peptide—AMC substrates of cathepsin B. These substrates contained a C-terminal 7-amino-4-methylcoumarin (AMC) reporter group and an N-terminal carboxybenzyl (Z) group. (FIG. 1c) Design of pH-selective peptidic inhibitors. Peptide—AOMK inhibitors were synthesized based on the AMC substrates that have high selectivity for cleavage at either pH 7.2 or pH 4.6.

FIG. 2 shows Cathepsin B activity at pH 7.2 and pH 4.6. Cathepsin B was preincubated at pH 7.2 or pH 4.6 at RT (27° C.) or at 37° C. for 30 min to 4 h. Z-Phe-Arg— AMC substrate (40 μM) was then added and proteolytic activity was monitored by measurement of AMC fluorescence. Activity is expressed relative to control cathepsin B with no preincubation (100%), data are displayed as the mean±SEM (n=4).

FIGS. 3(a)-3(b) show cathepsin B peptide cleavage analyses illustrate major dipeptidyl carboxypeptidase activity at pH 7.2 and pH 4.6, demonstrated by MSP-MS. (FIG. 3a) Cleavage at peptide bonds no. 1-13 of 14-mer peptide library substrates by cathepsin B. Cathepsin B cleavage of the 228 14-mer peptide library at pH 7.2 and pH 4.6 was evaluated as the number of cleavages occurring at each of the peptide bonds no. 1-13, that were generated at pH 7.2 and pH 4.6. (FIG. 3b) Time-dependent cleavage at position of peptide substrates at pH 7.2 and pH 4.6. The number of cleavages by cathepsin B at peptide bond no. 10 at 15 and 60 min incubation is shown. The time-dependent increase in the number of cleavages at position no. 10 may be consistent with sequential dipeptidyl carboxypeptidase cleavages.

FIGS. 4(a)-4(d) show differential cathepsin B substrate cleavage preferences at neutral pH 7.2 compared to acidic pH 4.6. (FIG. 4a) pH-dependent cleavage preferences of cathepsin B at pH 7.2 illustrated by IceLogo. IceLogo analysis demonstrates the relative frequency of amino acids at the P2, P1, P1′, and P2′ positions that surround the cleavage site (purple arrow). Residues shown in gray were not found at the indicated position. The amino acid described with lowercase “n” corresponds to norleucine, a sulfur-free isostere of methionine. Residues colored in red or blue were used in design of selective dipeptide—AMC substrates for pH 7.2 and pH 4.6, respectively. (FIG. 4b) pH-dependent cleavage preferences at pH 4.6 illustrated by IceLogo. IceLogo shows the preferred residues for the P2 to P2′ positions for cleavages occurring at pH 4.6. IceLogo features are described in the panel a description. (FIG. 4c) Dipeptide—AMC substrates selective for cathepsin B activity at pH 7.2 or pH 4.6. Based on MSP-MS peptide cleavage data for the preferred P2 and P1 residues adjacent to cleavage sites, peptide—AMC substrates selective for pH 7.2 and for pH 4.6 were designed and synthesized. Cathepsin B specific activities with each of the peptide—AMC substrates (40 μM final concentration) were assessed at pH 7.2 (red bars) and pH 4.6 (blue bars). (FIG. 4d) Ratios of cathepsin B specific activities at pH 7.2 and pH 4.6. The ratios of cathepsin B specific activity for pH 7.2/pH 4.6 and for pH 4.6/pH 7.2 are shown. Peptide—AMC substrates with a high ratio of pH 7.2/pH 4.6, and high ratio of pH 4.6/pH 7.2, were selected for modification by AOMK for inhibitor development.

FIGS. 5(a)-5(c) show dipeptide—AMC substrates that selectively monitor cathepsin B activity at neutral pH 7.2 compared to acidic pH 4.6, illustrated by kcat/Km values. (FIG. 5a) Z-Arg-Lys—AMC, pH 7.2 selective substrate. Cathepsin B activity with Z-Arg-Lys—AMC substrate at pH 7.2 and pH 4.6 was evaluated over a concentration range of 2.6 μM to 225 μM. (FIG. 5b) Z-Glu-Lys—AMC, pH 4.6 selective substrate. Cathepsin B activity was assessed with Z-Glu-Lys—AMC substrate at pH 7.2 and pH 4.6. (FIG. 5c) Z-Phe-Arg—AMC, substrate for both pH 7.2 and pH 4.6. Cathepsin B activity with Z-Phe-Arg—AMC substrate, a commonly used substrate, (77-79) at pH 7.2 and pH 4.6.

FIG. 6 shows cathepsin B pH-selective substrates Z-Arg-Lys—AMC and Z-Glu-Lys—AMC and the non-pH-selective substrate Z-Phe-Arg—AMC. The pH profiles of cathepsin B activity with the substrates Z-Arg-Lys—AMC, Z-Glu-Lys—AMC, and Z-Phe-Arg—AMC were assessed at pH 2 to 9, with substrate concentrations at 60 μM. Data points are shown as the mean±SEM (n=3). The pH curves are also illustrated for cathepsin B activity expressed as AMC RFU/s.

FIGS. 7(a)-7(b) show Z-Arg-Lys—AOMK and Z-Glu-Lys—AOMK inhibitors of cathepsin B at neutral pH compared to acidic pH conditions. (FIG. 7a) Z-Arg-Lys—AOMK inhibitor. Z-Arg-Lys—AOMK inhibition of cathepsin B was assessed at different inhibitor concentrations to determine IC50 values at pH 7.2 and pH 4.6. Z-Phe-Arg—AMC was used as substrate for cathepsin B assays. (FIG. 7b) Z-Glu-Lys—AOMK inhibitor. The inhibitor Z-Glu-Lys—AOMK at different concentrations was assessed for IC₅₀ values at pH 4.6 and pH 7.2.

FIG. 8(a)-8(b) shows Z-Arg-Lys—AOMK selectively inhibits cathepsin B cleavage of peptide substrates at pH 7.2 compared to pH 4.6, assessed by MSP-MS. (FIG. 8a) Z-Arg-Lys—AOMK (64 nM) at pH 7.2 inhibits cathepsin B cleavage of peptide library substrates assessed by MSP-MS. The inhibitor concentration was selected for —90% to inhibition at pH 7.2 (using Z-F-R—AMC substrate), which consisted of 64 nM Z-Arg-Lys— AOMK (93% inhibition at pH 7.2). MSP-MS assays analyzed the cleavage products generated from the peptide library by LC-MS/MS identification and quantification. The relative quantities of each peptide product generated in the absence of inhibitor or in the presence of inhibitor were plotted as the fold-change of each peptide product relative to no enzyme activity control. (FIG. 8b) Z-Arg-Lys—AOMK (64 nM) at pH 4.6 does not inhibit cathepsin B cleavage of peptide library substrates assessed by MSP-MS. Cathepsin B was incubated without and with the inhibitor at pH 4.6 for MSP-MS analyses of peptide products. The MSP-MS procedure and inhibitor concentrations are described in the panel a description.

FIG. 9 shows Model of Z-Arg-Lys—AOMK binding to cathepsin B at neutral pH 7.2: interaction of enzyme G1u245 with P2 Arg. (a) Model of the Z-Arg-Lys—AOMK inhibitor docking to cathepsin B at pH 7.2. Modeling of Z-Arg-Lys—AOMK binding to the active site of the cathepsin B structure is illustrated, achieved by the MOE software using the cathepsin B structure of PDB 1QDQ as template for analyses at pH 7.2. (61) The P1 Lys residue of Z-Arg-Lys—AOMK interacts with the enzyme S1 subsite, shown in the blue region. The P2 Arg residue of the inhibitor interacts with the enzyme S2 subsite region, shown in orange. The inhibitor AOMK warhead docking to the enzyme region corresponds to the S1′ and S2′ subsites, shown in gray. (b) Two-dimensional illustration of Z-Arg-Lys—AOMK and cathepsin B binding interactions at pH 7.2. The peptidic Z-Arg-Lys—AOMK inhibitor interacts with the active site of cathepsin B, modeled by MOE. The P2 Arg residue of the Z-Arg-Lys—AOMK shows a strong polar interaction with the Glu245 carboxylate of the S2 pocket of the enzyme. The P1 Lys and P2 Arg residues of the inhibitor interact with the corresponding Si and S2 subsites of the cathepsin B enzyme. The P1 Lys interacts with Glu122 and Asn72 of the S1 subsite. (60,68) The AOMK warhead resides within 3.75 Å from the catalytic Cys29 nucleophile, suggesting a binding mode for irreversible inhibition; the AOMK group occupies the S1′ region near the occluding loop. The Z group (benzyloxycarbonyl) appears partially solvent exposed and extended from the S2 region.

FIGS. 10(a)-10(b) show Z-Arg-Lys—AOMK inhibition of cathepsin B in human neuroblastoma cells. (a) Cell homogenates assayed for cathepsin B activity in the presence of inhibitors. Homogenates of SHSY5Y human neuroblastoma cells were prepared as described in the Methods and Materials. Cathepsin B activity in the homogenate was assayed with Z-Arg-Arg—AMC substrate in the presence of Z-Arg-Lys—AOMK or CA-074 (1 FAM each). Assays were conducted at high concentrations of inhibitors to completely inhibit activity; assays were conducted at pH 5.5 because this is a routine pH used to assay this enzyme in the literature. (71,72) Cathepsin B activity was expressed as nmol AMC/(m/min), mean±SD (*p<0.05 by Student's t test, n=3). (b) Cells incubated with inhibitors and assay of cathepsin B activity. Human neuroblastoma cells were incubated with Z-Arg-Lys—AOMK or CA-074Me (50 μM each) for 6 h at 37° C. Cells were homogenized as described in the Methods and Materials, and cathepsin B was assayed with Z-Arg-Arg—AMC substrate. Cathepsin B activity was expressed as nmol AMC/(m/min) and shown as mean±SD (*p<0.05 by Student's t test, n=6).

### DETAILED DESCRIPTION

In embodiments, the invention provides peptidic inhibitors of cathepsin B, and methods of use and preparation thereof, comprising an amino-terminally capped di-peptide Arg-Lys carboxy-terminally conjugated to a cysteine protease inhibitor. In embodiments the peptidic inhibitor of cathepsin B inhibits cathepsin B activity at neutral pH more effectively than at lower pH.

In embodiments, the invention provides that the cysteine protease inhibitor is acyloxymethyl ketone (AOMK). In embodiments, the invention provides that the cysteine protease inhibitor is alternatively selected from another electrophilic warhead that irreversibly reacts with the active site of cysteine proteases, including epoxysuccinates, vinyl esters, vinyl sulfones, allyl sulfones, vinyl sulfonates, hydroxymethylketone, diazomethylketones, aryloxymethylketones, sulfonium methylketones, fluoromethylketones or chloromethylketones (95). In addition, the invention provides that the cysteine protease inhibitor is selected from a reversible cysteine reactive warhead that includes aldehydes, thiomethylketones, oxymethylketones, cyclic ketones, amidomethylketones, nitriles, or various 1,2-dicarbonyl motifs (96).

In embodiments, the invention provides that the peptidic inhibitor of cathepsin B has an amino-terminal cap that is a benzyloxycarbonyl (Z). In embodiments, the amino-terminal cap is alternatively an acetyl (Ac), benzoyl (Bz), benzyl (Bzl), tert-butyloxycarbonyl (Boc), pyrazinylcarbonyl, cinnamoyl, naphthalene (Nap), fluorenylmethoxycarbonyl (Fmoc), pyrene (Pyr), phenothiazine (PTZ), morpholinyl, trifluoroacetamide, tosyl or other N-terminal protecting groups (97, 98).

In embodiments, the invention provides that the peptidic inhibitor of cathepsin B has a formula Z-Arg-Lys-AOMK (Z-R-K-AOMK).

In embodiments, the invention provides pharmaceutical compositions comprising the peptidic inhibitor of cathepsin B as described herein and a pharmaceutically acceptable excipient.

In embodiments, the invention provides methods of inhibiting neutral pH cathepsin B activity, comprising administering to a mammalian subject in need an effective amount of a peptidic inhibitor of cathepsin B comprising an amino-terminally capped di-peptide Arg-Lys carboxy-terminally conjugated to a cysteine protease inhibitor, wherein the peptidic inhibitor of cathepsin B inhibits cathepsin B activity at neutral pH more effectively than at lower pH.

In embodiments, the neutral pH cathepsin B activity is due to lysosomal leakage of cathepsin B to neutral cytosol, nucleus, plasma membrane or extracellular locations.

In embodiments, the invention provides methods for treatment of a disease or disorder characterized by lysosomal leakage of cathepsin B, comprising administering to a mammalian subject in need an effective amount of a pharmaceutical composition comprising a peptidic inhibitor of cathepsin B comprising an amino-terminally capped di-peptide Arg-Lys carboxy-terminally conjugated to a cysteine protease inhibitor, wherein the peptidic inhibitor of cathepsin B inhibits cathepsin B activity at neutral pH more effectively than at lower pH.

In embodiments, the invention provides the above methods wherein the cysteine protease inhibitor is acyloxymethyl ketone (AOMK). In embodiments, the invention provides the above methods wherein amino-terminal cap is a benzyloxycarbonyl (Z). In embodiments, the invention provides the above methods wherein the peptidic inhibitor of cathepsin B comprises the formula Z-Arg-Lys-AOMK (Z-R-K-AOMK).

In embodiments, the disease or condition is neurological. In embodiments, the neurological disease or condition is Alzheimer's disease (12-17), traumatic brain injury (TBI) (12, 18, 19), neurodegenerative conditions (20-25), TBI and ischemia (18-21), Parkinson's disease (22, 23) or other neurodegenerative condition or behavioral deficit.

In embodiments, the disease or condition is inflammatory, infectious or metabolic. In embodiments, the inflammatory or infectious disease or condition is atherosclerosis (28, 29), mucopolysaccaridosis, pancreatitis, Mycobacterium tuberculosis, dengue virus (26-32), pancreatitis (30, 31), pneumoccal meningitis (85), rheumatoid arthritis (48), osteoarthritis (101) or HIV (99).

In embodiments, the disease or condition is metabolic, such as Niemann-Pick disease or lysosomal storage disorders (24, 25).

In embodiments, the disease is cancer. In embodiments, the cancer is colorectal cancer or breast cancer (43-47) (due to extracellular cathepsin B) or thyroid cancer (50) (due to nuclear cathepsin B).

In embodiments, the condition is characterized by cytosolic cathepsin B initiated apoptotic cell death or activation of inflammatory IL-1beta production.

In embodiments, the disease or condition occurs at a neutral pH site in the subject, including nuceli, plasma membrane (100) or in the extracellular space.

In embodiments, the invention provides methods of producing pH-selective peptide-AMC substrates and novel peptidic-AOMK inhibitors of cathepsin B, as described herein.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.): Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within 10 that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself In embodiments, “about” can be used to mean, for example, within 10% of the recited value, within 5% of the recited value, or within 2% of the recited value.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals. The invention contemplates any effective route of administration, including peritoneal (e.g., i.v., i.m, s.c.), oral, mucosal, respiratory, and transdermal routes.

As used herein the term “pharmaceutically acceptable carrier” refers to an excipient, diluent, preservative, solubilizer, emulsifier, adjuvant, and/or vehicle with which the active compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincoft, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention, In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

### EXAMPLES

Cathepsin B is active at the neutral pH 7.2 (9, 10) of the cytosol (52, 53), as well as at the acidic pH 4.6 (6-8) within lysosomes (4, 52). The 400-fold difference in proton concentration at pH 4.6 compared to pH 7.2 alters the charge state of cathepsin B (12) and its substrates. These distinct pH conditions lead to the hypothesis that cathepsin B may possess different substrate cleavage preferences at cytosolic neutral pH compared to lysosomal acidic pH. We tested this hypothesis by comparing the substrate cleavage properties of cathepsin B at both pH conditions by global Multiplex Substrate Profiling by Mass Spectrometry (MSP-MS) using a peptide substrate library consisting of 228 peptides designed to contain diverse protease cleavage sites (54, 55). Furthermore, MSP-MS directly assesses the location of each cleavage site and can, therefore, distinguish aminopeptidase (56, 57), endopeptidase (58, 59), and carboxypeptidase activities (54, 60). Results showed that cathepsin B displays pH-selective cleavage properties represented by its prominent dipeptidyl carboxypeptidase activity and modest endopeptidase activity.

Based on the distinct cathepsin B cleavage properties at neutral pH compared to acidic pH conditions, peptide-AMC substrates and novel peptidic-AOMK inhibitors of cathepsin B were designed and evaluated for pH selectivity. Notably, Z-Arg-Lys-AOMK was revealed as a potent and selective inhibitor of neutral pH 7.2 cathepsin B activity. This inhibitor displayed high specificity for cathepsin B compared to other lysosomal cysteine cathepsins. These results demonstrate that the unique pH-dependent cleavage properties of cathepsin B provide the basis for design of Z-Arg-Lys-AOMK as a neutral pH inhibitor of cathepsin B. These findings suggest that neutral pH cathepsin B represents a unique form of the enzyme compared to the normal lysosomal cathepsin B.

Strategy to assess cleavage properties of cathepsin B for design of a neutral pH selective inhibitor. The workflow used to analyze cathepsin B cleavage properties for development of a neutral pH inhibitor is illustrated in FIG. 1. Unbiased MSP-MS assays evaluated the cleavage properties of cathepsin B at neutral pH 7.2 and acidic pH 4.6 using a peptide library consisting of 228 peptide substrates (14 residues in length) containing 2,964 diverse cleavage sites. Cathepsin B cleavage products were identified and quantified by nano-liquid chromatography tandem mass spectrometry (nano-LC-MS/MS) to determine the frequencies of amino acid residues adjacent to cleavage sites at P1-↓P1′ residues. Preferred residues at the P2 and P1 positions were utilized for design of dipeptide fluorogenic substrates. Substrate sequences that were selectively hydrolyzed by cathepsin B at pH 7.2 or 4.6 were synthesized with the acyloxymethyl ketone (AOMK) warhead to generate peptidic inhibitors. This approach demonstrates that a pH-selective inhibitor of cathepsin B can be rationally designed from substrate sequence properties.

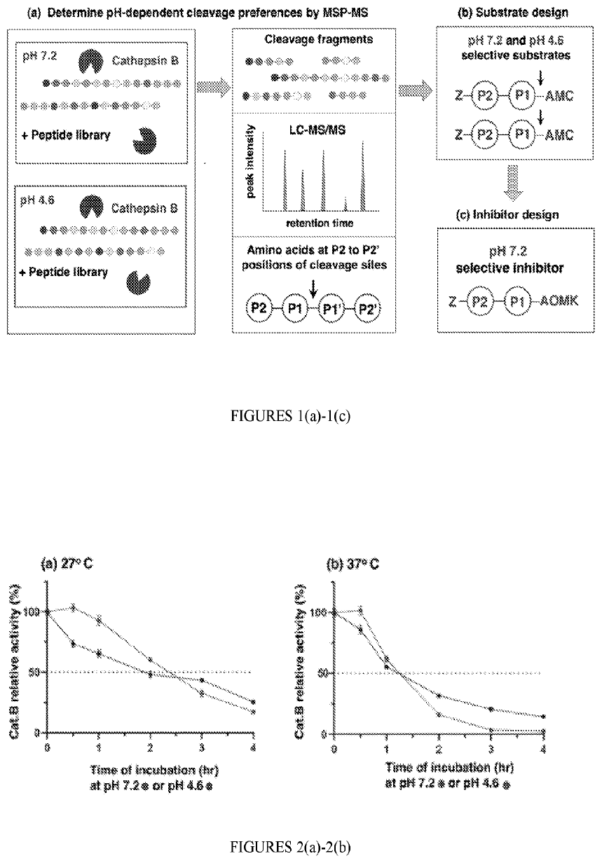

Cathepsin B stability at neutral pH 7.2 and acidic pH 4.6. Prior to determining the substrate cleavage profiles of cathepsin B, we evaluated enzyme stability at pH 7.2 and pH 4.6 by pre-incubating the enzyme for up to 4 hours at room temperature (RT, 27° C.) and at 37° C., followed by assays with Z-Phe-Arg-AMC substrate. After 1 hour of pre-incubation, the relative activity at each pH and temperature was above 50%, and decreased with longer pre-incubation times (FIG. 2). We, therefore, performed the MSP-MS cleavage assays at RT for up to 1 hour to generate data for active enzyme. These in vitro assays show that cathepsin B is active at both pH 7.2 and pH 4.6, and represent a model for studying cathepsin B activity.

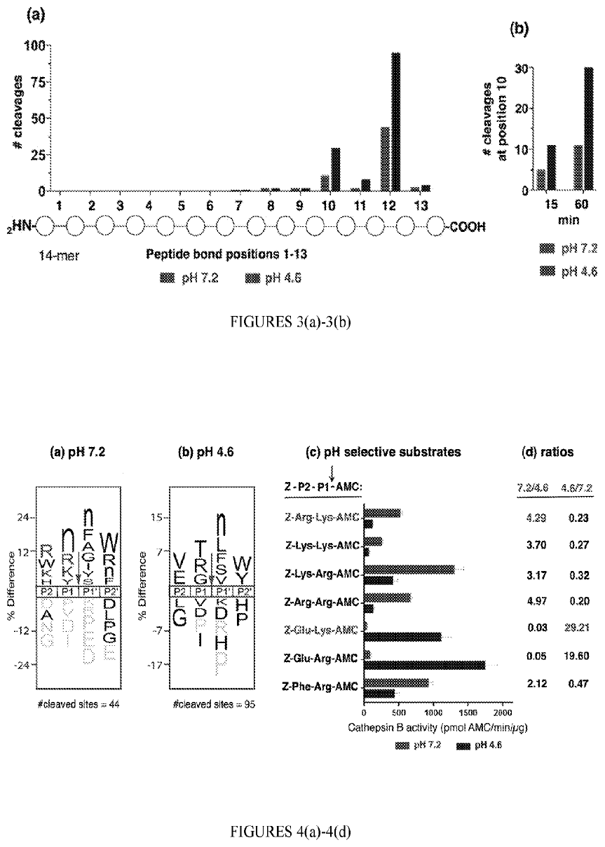

Cleavage profile of cathepsin B demonstrates strong dipeptidyl carboxypeptidase specificity. Human recombinant cathepsin B was incubated with the 228 14-mer library for 60 minutes followed by nano-LC-MS/MS and PEAKS bioinformatics to quantify peptide products. At pH 7.2 cathepsin B cleaved 66 peptide bond sites, and at pH 4.6 the enzyme cleaved 142 sites (data not shown). Cleavage was defined by peptide products having intensity values that were at least 8-fold above that in the denatured enzyme control, based on the criteria to minimize false positive rate (data not shown). The distribution of cleavages at each of the 13 peptide bonds among the peptide substrates was quantified and proteolysis was found to occur primarily at position #12 indicating dipeptidyl carboxypeptidase activity (FIG. 3a). Cleavage at position #10 was also prevalent. Fewer numbers of cleavages occurred at positions #7-9 and #11 that may represent endopeptidase cleavages, and no cleavages were observed at positions #1-6.

The presence of prominent dipeptidyl carboxypeptidase activity suggested that sequential cleavage at position 12 followed by cleavage at position 10 may occur in a time-dependent manner. Evaluation of the number of cleavages occurring at position 10 at 15 min and 60 min found that increases occurred in a time-dependent manner (FIG. 3b), consistent with dipeptidyl carboxypeptidase processing at position 12 followed by such cleavages at position 10 (data not shown). These findings illustrate the primary exopeptidase activity of cathepsin B as a dipeptidyl carboxypeptidase, with low endopeptidase activity, at both neutral and acidic pH conditions.

pH-dependent cleavage properties of cathepsin B. Cathepsin B displayed differences in cleavage preferences at pH 7.2 compared to pH 4.6. The frequencies of amino acid residues located at positions P2-P1-↓P1′-P2′ to generate cleaved peptide products were analyzed by MSP-MS for the major cleavages occurring as dipeptidyl carboxypeptidase cleavages at position 12. IceLogo schematically illustrates the relative frequency of amino acid residues occurring at P2-P1-↓P1′-P2′ residues at pH 7.2 and 4.6 (FIG. 4a, b).

At the P1 position, cathepsin B at pH 7.2 preferred the basic residues Arg and Lys, along with norleucine and Tyr (FIG. 4a). At pH 4.6, the P1 positions displayed preferences for the non-charged Thr and Gly residues, as well as the basic residue Arg (FIG. 4b).

At the P2 position, differences in the preferences for negative and positive residues were observed at pH 7.2 and pH 4.6 (FIG. 4a, b). At pH 4.6, the acidic Glu residue was a preferred residue at the P2 position, as well as hydrophobic Val. In contrast, at pH 7.2, the basic residues Arg, Lys, and His were preferred at the P2 position, as well as Trp. These preferred P2 residues appear consistent with the presence of Glu245 at the S2 pocket of the protease that interacts with the P2 residue of the cathepsin B substrate (61). At pH 4.6, the uncharged Glu245 could interact with the uncharged Glu as the P2 residue, while at neutral pH 7.2 the negatively charged Glu245 would be amenable to interacting with the positively charged P2 basic Arg or Lys residues. These preferred residues at the P2 positions may be informative for design of pH-selective substrates of cathepsin B.

Development of pH selective peptide-AMC substrates for cathepsin B. The MSP-MS substrate profiling results provided the basis for design of pH selective dipeptide-AMC substrates (FIG. 4c). A series of pH 7.2 selective substrates were designed with basic residues at the P2 position, consisting of Z-Arg-Lys-AMC, Z-Lys-Lys-AMC, Z-Lys-Arg-AMC, and Z-Arg-Arg-AMC. At pH 4.6, the preference for Glu at the P2 position was used for design of the pH 4.6 selective substrates Z-Glu-Lys-AMC and Z-Glu-Arg-AMC.

Evaluation of pH substrate selectivity found that dipeptide substrates with basic residues at both P2 and P1 positions were more rapidly cleaved at neutral pH 7.2 than at pH 4.6 by cathepsin B; these substrates consisted of Z-Arg-Lys-AMC, Z-Lys-Lys-AMC, Z-Lys-Arg-AMC, and Z-Arg-Arg-AMC (FIG. 4c). Z-Arg-Lys-AMC and Z-Arg-Arg-AMC had the highest ratio of pH 7.2/pH 4.6 activities (FIG. 4d). Furthermore, acid pH preferring substrates consisted of Z-Glu-Lys-AMC and Z-Glu-Arg-AMC with Glu at the P2 position (FIG. 4c). Z-Glu-Lys-AMC displayed the highest ratio of pH 4.6/pH 7.2 activities, indicating preference for pH 4.6 cathepsin B activity (FIG. 4d).

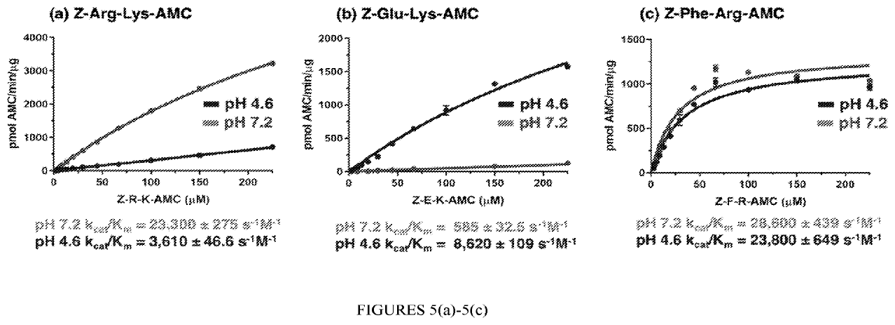

Substrate concentration studies showed that at pH 7.2, cathepsin B displayed preference for the Z-Arg-Lys-AMC substrate, shown by the greater rate of hydrolysis of this substrate at pH 7.2 compared to pH 4.6, as illustrated by kcat/Km values (FIG. 5a). At pH 4.6, cathepsin B preferred the Z-Glu-Lys-AMC substrate (FIG. 5b), shown by the more rapid rate of hydrolysis at pH 4.6 over pH 7.2. In contrast, Z-Phe-Arg-AMC was hydrolyzed at similar rates by cathepsin B at both pH 4.6 and 7.2 (FIG. 5c).

The complete pH profiles were assessed for the pH-selective substrates Z-Arg-Lys-AMC and Z-Glu-Lys-AMC, and the non-pH selective substrate Z-Phe-Arg-AMC (FIG. 6). Hydrolysis of Z-Arg-Lys-AMC was maximal at pH 7.8, with >50% activity occurring between pH 6.2 to pH 8.5, indicating Z-Arg-Lys-AMC as a selective neutral pH substrate of cathepsin B. In contrast, Z-Glu-Lys-AMC was optimally hydrolyzed at pH 4.6, with >50% activity occurring at pH 3.6 to pH 5.6, indicating Z-Glu-Lys-AMC as a selective acidic pH substrate. Z-Phe-Arg-AMC was hydrolyzed across a wide pH range with 50% of the maximum activity occurring between pH 3.8 and 8.6. These data clearly show that cathepsin B has distinct enzymatic properties at pH 4.6 and pH 7.2, and these differences can be exploited by rational design of pH selective substrates.

The peptidic substrates were assessed for specificity for related lysosomal cysteine cathepsin proteases. Z-Arg-Lys-AMC selectively monitored cathepsin B activity primarily at pH 7.2, and showed no activity for cathepsin L or cathepsin V at pH 7.2 (data not shown). Z-Glu-Lys-AMC was selective for cathepsin B activity at pH 4.6, and displayed no activity for cathepsins L or V. In contrast, Z-Phe-Arg-AMC was hydrolyzed by cathepsin B at both pH conditions, and this substrate was also cleaved by cathepsins L and V at pH 4.6.

Z-Arg-Lys-AOMK and Z-Glu-Lvs-AOMK inhibitors of cathepsin B. The strategy to incorporate the AOMK warhead to replace the AMC group of the peptide-AMC substrates (57, 62-64) was utilized to design and synthesize the Z-Arg-Lys-AOMK and Z-Glu-Lys-AOMK peptidic inhibitors (data not shown).

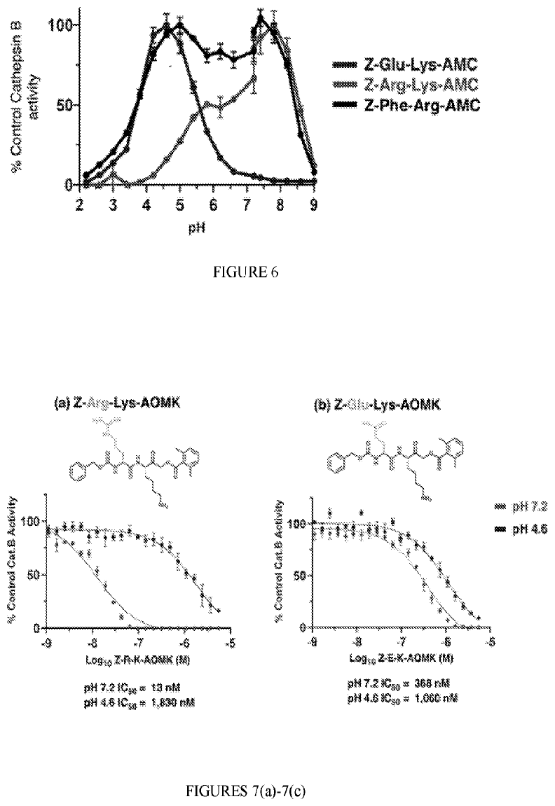

Z-Arg-Lys-AOMK displayed selective inhibition of cathepsin B at pH 7.2 compared to pH 4.6 (FIG. 7a and Table 1). Determination of kinetic constants showed that Z-Arg-Lys-AOMK was a potent inhibitor with KI value of 130 nM at pH 7.2, but was less effective at pH 4.6 with a KI of 15,000 nM at pH 4.6 (Table 1). The KI values show that this inhibitor displays 115-fold greater potency at pH 7.2 compared to pH 4.6. The kinact/KI constant was 1.1×105 M⁻¹s⁻¹ at pH 7.2, and 1.8×103 M⁻¹s⁻¹ at pH 4.6 (Table 1). The inhibitory effectiveness of Z-Arg-Lys-AOMK was also illustrated by its low IC50 value of 20 nM, compared to its lower effectiveness at pH 4.6 with IC50 value of 1,500 nM. These kinetic studies illustrate Z-Arg-Lys-AOMK as a potent neutral pH inhibitor of cathepsin B.

Compared to Z-Arg-Lys-AOMK, Z-Glu-Lys-AOMK displayed less effective inhibition of cathepsin B at both pH 7.2 and pH 4.6 (FIG. 7b and Table 1). Kinetic analyses showed that Z-Glu-Lys-AOMK had K_{I} values of 2,300 nM and 7,900 nM to at pH 7.2 and pH 4.6, respectively (Table 1). The k_{inact}/Kᵢ values for this inhibitor were 8.2×10³ M⁻¹s⁻¹ and 2.0×10³ M⁻¹s⁻¹ at pH 7.2 and pH 4.6, respectively (Table 1). These data showed that Z-Glu-Lys-AOMK was about 3.5-fold more potent at pH 7.2 compared to pH 4.6, with Kₜ values for both pHs at micromolar levels. IC₅₀ values of 320 nM and 1,100 nM for pH 7.2 and 4.6,

TABLE 1
Kinetic properties of Z-Arg-Lys-AOMK
and Z-Glu-Lys-AOMK inhibitors

| Kinetic | Z-Arg-Lys-AOMK | | Z-Glu-Lys-AOMK | |
|---|---|---|---|---|
| constant | pH 4.6 | pH 7.2 | pH 4.6 | pH 7.2 |
| K_{I} (nM) | 15,000 ± 6,000 | 130 ± 50 | 7,900 ± 830 | 2,300 ± 620 |
| K_{inact}/K_{I} | 1.8 × 10³ | 110 × 10³ | 2.0 × 10³ | 8.2 × 10³ |
| (M⁻¹s⁻¹) | | | | |
| IC₅₀ (nM) | 1,500 ± 650 | 20 ± 8.3 | 1,100 ± 480 | 320 ± 45 |

K_{I}, K_{inact}/K_{I}, and IC₅₀ values for the irreversible inhibitors of cathepsin B were determined as explained in the methods. k_{obs} constants were determined by plots of cathepsin B activity in time courses with different inhibitor concentrations with curve fitting Y = Y₀*e^{(-k}_{obs}^{*X)}, where Y₀ is the activity for the control with no inhibitor condition, Y is the activity in the presence of inhibitor, X is time. K_{I} and k_{inact} values were calculated from k_{obs} values with the equation k_{obs} = k_{inact}*[I]/(K_{I} + [I]) (graphs shown in supplemental FIG. S6), where [I] is inhibitor concentration, and K_{I} is the inhibitor concentration (x-axis) where y = k_{inact}/2 and k_{inact} is the maximum rate of inactivation at saturating inhibitor concentrations. K_{I} and IC₅₀ values are expressed as the mean ± SD (n = 4, n = 6, respectively)

respectively, were of similar orders of magnitude. The micromolar levels of Z-Glu-Lys-AOMK for inhibition at both pHs were less effective than the nanomolar levels of Z-Arg-Lys-AOMK for neutral pH inhibition of cathepsin B.

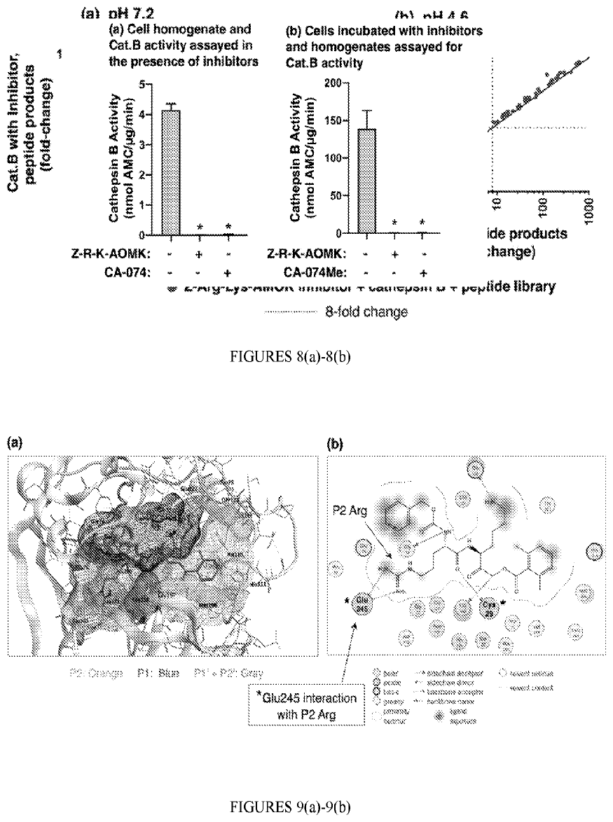

Neutral pH selective inhibition of peptide library cleavages by Z-Arg-Lys-AOMK. To further validate the neutral pH selectivity of Z-Arg-Lys-AOMK inhibition, cathepsin B was pre-incubated with this inhibitor at 64 nM at pH 7.2 and pH 4.6, and proteolytic activity was assessed using the 228-member peptide library in MSP-MS assays. The 64 nM concentration of Z-Arg-Lys-AOMK was chosen because it reduced cathepsin B activity with Z-Phe-Arg-AMC as substrate by 93% at pH 7.2 and by 5% at pH 4.6 (FIG. 8). At pH 7.2, Z-Arg-Lys-AOMK completely inhibited peptide cleavages by cathepsin B after 1 hr incubation (FIG. 8a). However, at pH 4.6, Z-Arg-Lys-AOMK (64 nM) did not inhibit cathepsin B formation of peptide products (FIG. 8b). These findings show that Z-Arg-Lys-AOMK selectively inhibits cathepsin B cleavage of peptides at neutral cytosolic pH compared to acidic lysosomal pH conditions.

Irreversible mechanism of Z-Arg-Lys-AOMK and Z-Glu-Lys-AOMK inhibitors. The irreversible mechanism of the inhibitors was demonstrated by pre-incubation of each inhibitor with cathepsin B, followed by dilution and activity measurements (data not shown). Control enzyme without inhibitor displayed a linear time-dependent progression of proteolytic activity. Pre-incubation with Z-Arg-Lys-AOMK or Z-Glu-Lys-AOMK at pH 7.2 and pH 4.6, respectively, resulted in no cathepsin B activity after dilution of the inhibitors, indicating the irreversible mechanism of these inhibitors.

Z-Arg-Lys-AOMK and Z-Glu-Lys-AOMK specifically inhibit cathepsin B compared to other cysteine cathepsins. At pH 7.2, Z-Arg-Lys-AOMK inhibited cathepsin B with a IC50 of 20 nM, which was more potent by 22-fold, 110-fold, and 43-fold than cathepsin V (IC50=440 nM), cathepsin S (IC50=2,200 nM), and cathepsin C (IC50=850 nM) inhibition (Table 2). At pH 7.2, cathepsins K and H were minimally inhibited by Z-Arg-Lys-AOMK at 16 μM. At pH 4.6, Z-Arg-Lys-AOMK inhibited cathepsin B with IC50 of 1,500 nM, and at 16 μM this inhibitor showed minimal inhibition of cathepsins L, V, S, X, C, and no inhibition of cathepsin K or cathepsin H.

TABLE 2
Specificity of Z-Arg-Lys-AOMK and Z-Glu-Lys-AOMK for inhibition
of cathepsin B compared to other cysteine cathepsins

| | Z-Arg-Lys-AOMK | | Z-Glu-Lys-AOMK | |
|---|---|---|---|---|
| | IC₅₀ (nM) | | IC₅₀ (nM) | |
| Protease | pH 4.6 | pH 7.2 | pH 4.6 | pH 7.2 |
| Cathepsin B | 1,500 | 20 | 1,100 | 320 |
| Cathepsin C | >16,000 | 850 | 8,600 | 12,000 |
| | (6% inhibition) | | | |
| Cathepsin H | — | >16,000 | — | — |
| | | (29% Inhibition) | | |
| Cathepsin K | — | >16,000 | >16,000 | — |
| | | (31% Inhibition) | (10% inhibiton) | |
| Cathepsin L | >16,000 | NA | >16,000 | NA |
| | (32% Inhibition) | | 23% Inhibiton | |
| Cathepsin S | >16,000 | 2,200 | >16,000 | >16,000 |
| | (13% inhibition) | | (26% inhibition) | (30% inhibition) |
| Cathepsin V | >16,000 | 440 | 1,900 | >16,000 |
| | (63% inhibition) | | | (10% inhibition) |
| Cathepsin X | >16,000 | NA | — | NA |
| | (21% inhibition) | | | |

Inhibitors were evaluated for protease specificity among the 8 cysteine cathepsins, achieved by monitoring the activity of each enzyme in the presence of a range of inhibitor concentrations from nM to 16 μM (without pre-incubation). IC₅₀ values were generated for the inhibitors Z-Arg-Lys-AOMK and Z-Glu-Lys-AOMK for each of the cysteine cathepsin enzymes. IC₅₀ values are indicated as >16,000 nM when partial inhibition was observed (% inhibition is shown). No IC₅₀ values are indicated when there was no inhibition at 16,000 nM inhibitor (indicated by ‘—’). NA indicates that the enzyme had no activity at the indicated pH.

Z-Glu-Lys-AOMK also demonstrated specific inhibition of cathepsin B compared to other cysteine cathepsins (Table 2). At pH 7.2, the inhibitor was 38-fold more potent for cathepsin B (IC50=320 nM) relative to cathepsin C (IC50=12,000 nM). At pH 4.6, weak inhibition of cathepsin V (IC50=1,900 nM) and cathepsin C (IC50=8,600 nM) occurred while the other cathepsin enzymes tested were minimally inhibited or not inhibited by Z-Glu-Lys-AOMK (at 16 μM) at either pH condition.

These data illustrate the high specificity of Z-Arg-Lys-AOMK and Z-Glu-Lys-AOMK inhibitors for cathepsin B over other members of the cysteine cathepsin family.

Molecular docking of Z-Arg-Lys-AOMK to cathepsin B at neutral pH 7.2: interaction of Glu245 of the enzyme with P2 Arg. Modeling of Z-Arg-Lys-AOMK binding interactions to cathepsin B was assessed by the Molecular Operating Environment (MOE) software (65, 66), MOE generated a representation of inhibitor binding to the active site of human cathepsin B (PDB: 1QDQ) (61) at pH 7.2 consisting of P2 and P1 residues of the peptidic inhibitor interacting with the S2 and S1 subsites of the enzyme, according to the Schechter-Berger nomenclature (67) (FIG. 9). The P2 Arg residue of the Z-Arg-Lys-AOMK shows a strong polar interaction with the carboxylate of Glu245 in the S2 subsite of the enzyme. Glu245 at pH 7.2 is negatively charged (based on its pKa of 5.1 (68), which is predicted to interact with the positively charged P2 Arg of the inhibitor at neutral pH. The P1 Lys residue of the inhibitor interacts with Glu122 and Asn72 of the enzyme S1 pocket (61, 69). The AOMK warhead occupies the S1′ region near the occluding loop; furthermore, the AOMK carbon atom resides less than 3.4 Å from the catalytic Cys29 nucleophile, suggesting a binding mode for irreversible inhibition. The Z group (benzyloxycarbonyl) appears partially solvent exposed and extended from the S2 region. In contrast, a pH 4.6 model of Z-Arg-Lys-AOMK docking to cathepsin B showed a lack of Glu245 interaction with the Arg moiety of this inhibitor (data not shown). These features illustrate a model of Z-Arg-Lys-AOMK binding to the active site of cathepsin B at neutral pH

At pH 4.6, Z-Arg-Lys-AOMK docking by MOE modeling showed no interactions of Glu245 (of the enzyme) with the Arg of this inhibitor (data not shown), which contrasts with Glu245 interactions with Arg of Z-Arg-Lys-AOMK at pH 7.2. MOE modeling suggests Glu245 interaction with Z-Arg-Lys-AOMK at pH 7.2, but no interaction with Z-Glu-Lys-AOMK. MOE calculations of the inhibitor binding energies to cathepsin B at pH 7.2 and pH 4.6 show more favorable interactions of Z-Arg-Lys-AOMK at pH 7.2 compared to pH 4.6 (Table 3). These binding energies were estimated based on interactions of enzyme active site residues with the inhibitors.

TABLE 3
Binding energies of Z-Arg-Lys-AOMK and Z-Glu-Lys-AOMK
to cathepsin B at neutral pH 7.2 and acidic pH 4.6

Binding energy (kcal/mol)

| Inhibitor | pH 7.2 | pH 4.6 |
|---|---|---|
| Z-Arg-Lys-AOMK | −55.5 | −26.2 |
| Z-Glu-Lys-AOMK | −52.3 | −55.9 |

For Z-Arg-Lys-AOMK, the more negative binding energy calculated for pH 7.2 compared to pH 4.6 indicates a more favorable interaction of this inhibitor to cathepsin B at pH 7.2. These calculations are made with Glu245 is protonated at pH 4.6. For Z-Glu-Lys-AOMK, the equivalent binding energies calculated for pH 7.2 and pH 4.6 indicate a similar interaction of this inhibitor to cathepsin B at these two pH conditions.

With respect to Z-Glu-Lys-AOMK, MOE shows that Z-Glu-Lys-AOMK lacks interactions with the Glu245 of the S2 pocket of cathepsin B (data not shown). Furthermore, similar binding energies were calculated at pH 7.2 and pH 4.6 for Z-Glu-Lys-AOMK (Table 3), suggesting that this inhibitor was not pH selective. This lack of pH selectivity is supported by our inhibition studies (Table 1). The MOE modeling implicates the importance of Glu245 of cathepsin B for effectiveness of the neutral pH inhibitor Z-Arg-Lys-AOMK.

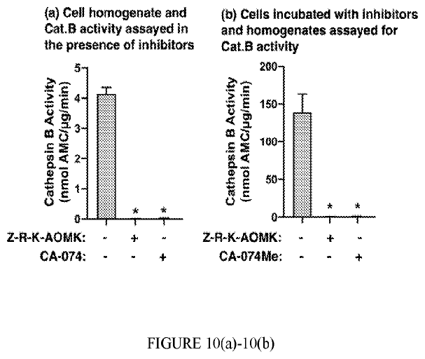

Z-Arg-Lys-AOMK inhibition of intracellular cathepsin B and cell permeability. Z-Arg-Lys-AOMK was evaluated for its ability to inhibit cathepsin B activity in human neuroblastoma cell lysate using Z-Arg-Arg-AMC substrate. Proteolytic activity in the cell lysate was completely inhibited by Z-Arg-Lys-AOMK (1 μM) and by CA-074 (1 μM), a specific inhibitor of cathepsin B (70) (FIG. 10a). These data show that Z-Arg-Lys-AOMK and CA-074 inactivate cathepsin B.

The cell permeability of Z-Arg-Lys-AOMK was evaluated by incubation of neuroblastoma cells with this inhibitor (50 μM) for 6 hrs. Cells were also incubated with CA-074Me (50 μM); CA-074Me is known to enter cells and converted by esterases to the potent CA-074 inhibitor of cathepsin B (71). After incubation, cells were washed, and homogenates assayed for cathepsin B activity with Z-Arg-Arg-AMC substrate. Z-Arg-Lys-AOMK and CA-074Me completely inhibited cellular cathepsin B activity (FIG. 10b). These results demonstrate that the Z-Arg-Lys-AOMK is cell permeable and inhibits intracellular cathepsin B.

This study designed and developed a neutral pH selective inhibitor, Z-Arg-Lys-AOMK, of cathepsin B based on the enzyme's distinct substrate cleavage properties observed at neutral pH 7.2 compared to acidic pH 4.6. Cathepsin B functions at the neutral pH locations of the cytosol and extracellular environments of brain disorders (12-25) and human diseases of different physiological systems (26-32), which contrasts with the normal location of cathepsin B in lysosomes of acidic pH. Development of the neutral pH inhibitor was based on the hypothesis that the unique pH-dependent cleavage properties of cathepsin B may provide the basis for the design of selective neutral pH inhibitors. Specifically, the differential cleavage properties of cathepsin B at neutral pH compared to acidic pH were revealed by MSP-MS substrate profiling which utilizes a peptide library containing all neighbor and near-neighbor amino acid combinations. MSP-MS assays revealed preferences of cathepsin B for residues at the P2 and P1 positions of the cleavage site (P1-↓P1′). The P2 position prefers Glu (E) at acidic pH, but prefers a basic residue Arg (R) at neutral pH. At the P1 position, basic residues are preferred at both neutral and acidic pHs. These cleavage properties led to the design of Z-Arg-Lys-AMC as a neutral pH 7.2 selective substrate, and Z-Glu-Lys-AMC as an acidic pH 4.6 selective substrate. Inhibitors were generated by synthesis of these dipeptide substrates with the AOMK (acyloxymethyl ketone) warhead. Z-Arg-Lys-AOMK was found to be an effective irreversible inhibitor of cathepsin B at neutral pH with nanomolar potency. Z-Arg-Lys-AOMK displays 100-fold more potent inhibition of cathepsin B at neutral pH compared to acidic pH. These findings indicate Z-Arg-Lys-AOMK as a neutral pH inhibitor of cathepsin B, thus, validating our hypothesis that substrate specificity differences can be utilized for rational design of pH selective inhibitors. Surprisingly, Z-Glu-Lys-AOMK was not selective for inhibition at acid pH; thus, addition of reactive warheads to peptide substrates may not always retain pH selectivity. Nonetheless, these findings demonstrate that pH selective inhibitors of cathepsin B can be developed based on its pH-dependent cleavage properties.

A notable finding of this study is that cathepsin B displays similar stability at both neutral pH 7.2 and acidic pH 4.6 conditions. While cathepsin B normally functions at the acidic pH 4.6 within lysosomes, we show that cathepsin B has similar stability at both neutral and acidic pH conditions. Cathepsin B was more stable at pH 7.2 than at pH 4.6 for up to 2 hours at room temperature (RT). Using conditions that maintain stability, cathepsin B activity in this study was conducted with RT incubation up to 60 min for MSP-MS assays and up to 30 min for fluorogenic assays. Studies at 37° C. were also conducted and showed similar stability of cathepsn B activity at pH 7.2 and pH 4.6. These stability studies complement reports in the field that cathepsin B becomes inactivated with time at neutral and alkaline conditions of pH 7.0-9.5 (52, 74-76). Our data provides new information that similar stability and inactivation properties of cathepsin B are observed at pH 7.2 and pH 4.6.

Cathepsin B is known to cleave folded protein substrates such as MARKS, collagen, and thyroglobulin (49, 77-79) via its endopeptidase activity. Using a substrate library of synthetic peptides that lack secondary structure, we can detect both endoprotease and exoprotease activity for any protease. We show that cathepsin B is primarily a dipeptidyl carboxypeptidase enzyme and sequential removal of dipeptides from the C-terminus was evident. Detection of dipeptidyl carboxypeptidase activity of protein substrates using traditional gel shift assays is difficult as the molecular weight changes are minor. However, using mass spectrometry, hydrolysis of peptide substrates into shorter products can be readily detected and quantified.

The role of the occluding loop for exopeptidase compared to endopeptidase activities of cathepsin B has been demonstrated by deletion mutagenesis of the loop domain, which resulted in the absence of exopeptidase activity and presence of only endopeptidase activity (80). Furthermore, site-directed mutagenesis of selected residues within the occluding loop resulted in increased endopeptidase activity (77). These studies indicate that the occluding loop regulates the exopeptidase and endopeptidase activities of cathepsin B.

Cathepsin B activity is typically monitored with Z-Phe-Arg-AMC and Z-Arg-Arg-AMC fluorogenic substrates for endopeptidase activity (82-84). However, use of extended peptide substrates that can be cleaved by either endopeptidases or exopeptidases in the MSP-MS cleavage analyses illustrated the predominant exopeptidase activity of cathepsin B at both neutral and acidic pHs. These data demonstrate that these commonly used peptide-AMC substrates monitor both the dipeptidyl carboxypeptidase and endopeptidase activities of cathepsin B.

The differential cleavage profiles of cathepsin B at neutral pH 7.2 and acidic pH 4.6 conditions by MSP-MS provided the basis for development of pH selective peptide-AMC substrates. At the P2 position, cathepsin B demonstrated preference for Glu at pH 4.6, but at pH 7.2 the enzyme preferred basic residues of Arg and Lys. At the P1 position, cathepsin B showed preference for basic residues Arg or Lys residues at acidic and neutral pH conditions. Prior studies of cathepsin B cleavage properties at pH 5.5 (72, 73) showed that the enzyme prefers P2 residues of Arg and Lys, but not Glu, and prefers P1 residues of Arg and Lys. These findings suggest that the cleavage specificity of cathepsin B at pH 5.5 (72, 73) resembles that of cathepsin B at pH 7.2. But cathepsin B at pH 5.5 did not display the pH 4.6 preference of the enzyme for Glu as the P2 residue found in this study. These findings together demonstrate pH-dependent cleavage specificities of cathepsin B.

The differential P2 and P1 residue preferences of cathepsin B were utilized to design and assess pH-selective peptide-AMC substrates. The Z-Arg-Lys-AMC substrate displayed high preference for neutral pH cathepsin B compared to several related substrates tested. The presence of Glu at the P2 position of Z-Glu-Lys-AMC was the rationale for its function as a selective substrate for acidic pH 4.6 cathepsin B. These findings demonstrate that the pH-dependent cleavage properties can provide the basis for design of pH-selective substrates of cathepsin B.

Significantly, design and synthesis of the Z-Arg-Lys-AOMK inhibitor resulted in selective and potent inhibition of neutral pH cathepsin B activity; this inhibitor was designed based on the neutral pH selective Z-Arg-Lys-AMC substrate. At pH 7.2, the Z-Arg-Lys-AOMK inhibitor displayed potent inhibition with a low Ki of 130 nM, but was less effective at pH 4.6 with a higher K_{I} of 15,000 nM. These results show that Z-Arg-Lys-AOMK is 115 times more potent at neutral pH 7.2 compared to acidic pH 4.6. Z-Arg-Lys-AOMK was shown to be an irreversible inhibitor with specificity for inhibition of cathepsin B over other cysteine cathepsins (cathepsins L, V, S, X, K, C, and H). Docking of Z-Arg-Lys-AOMK to cathepsin B at pH 7.2 was modeled by MOE which illustrated the active site binding features of the inhibitor. The model featured the ionic interaction of the positively charged P2 Arg residue of the peptidic inhibitor with the negatively charged Glu245 residue of the S2 subsite of cathepsin B at pH 7.2, which was absent at pH 4.6. This proposed interaction is consistent with studies showing the importance of Glu245 for interactions of the enzyme with the P2 residue of peptides (61, 69). Future studies of inhibitor and enzyme binding interactions can be gained through in-depth structural and computational investigation. Importantly, the findings of this study demonstrate Z-Arg-Lys-AOMK as a novel inhibitor that selectively and potently inhibits cathepsin B at neutral pH.

The Z-Glu-Lys-AOMK inhibitor displayed inhibition of neutral and acidic cathepsin B at micromolar levels of inhibitor with only a 3-fold difference in potency, even though the substrate Z-Glu-Lys-AMC preferentially detected acidic pH cathepsin B activity (rather than neutral pH activity). It appears that substitution of AOMK for the AMC of the Glu-Lys dipeptide removed its pH selectivity. It is noted that Lys at the P1 position is preferred for neutral pH cathepsin B activity, and, thus, the Lys at P1 may influence the inhibitor properties of Z-Glu-Lys-AOMK. These data show that a pH selective peptide-AMC substrate may not always lead to a pH selective peptidic AOMK inhibitor. Nonetheless, Z-Glu-Lys-AOMK represents a novel inhibitor of cathepsin B.

The neutral, cytosolic pH functions of cathepsin B due to lysosomal leakage occur in brain disorders and in numerous human diseases involving physiological organ systems (12-32). Lysosomal leakage results in translocation of cathepsin B from the lysosome to the cytosol where cathepsin B initiates apoptotic cell death (33-36) and activates IL-113 production in inflammation (37-40). Cathepsin B leakage to the cytosol occurs in brain disorders of AD (14-17), TBI and ischemia (18-21), Parkinson's disease (22, 23), Niemann-Pick disease and lysosomal storage disorders (24, 25), and pneumoccal meningitis (85). These neurodegenerative disorders also involve calpain in membrane permeabilization of lysosomes, allowing cathepsin B to exit the lysosome and enter the cytosol, known as the calpain-cathepsin hypothesis (86, 87). Other human diseases that involve lysosomal leakage of cathepsin B include autoinflammatory disease (26, 27), atherosclerosis (28, 29), and pancreatitis (30-32). In addition to the pathogenic function of cathepsin B in the cytosol, cathepsin B also functions at the neutral pH of extracellular locations in cancer (43-47), rheumatoid arthritis (48), nuclear locations in thyroid carcinoma (50), chromosome segregation (51), and in the thyroid follicle (49). In cancer, the tumor environment has been found to be at pH 6.8 due to the Warburg effect for tumor acidosis (88, 89); the Z-Arg-Lys-AOMK inhibitor was also found to be effective at pH 6.8 with an IC₅₀ value of 22 nM (data not shown). Overall, the prevalence of cytosolic cathepsin B in human diseases emphasizes the critical importance of this study to gain an understanding of the unique neutral pH properties of cathepsin B compared to its normal acidic lysosomal features.

In summary, the novel pH-dependent cleavage properties of the major to dipeptidyl carboxypeptidase activity of cathepsin B were revealed by MSP-MS substrate profiling that led to design of pH-selective substrates and novel peptidic AOMK inhibitors. Notably, Z-Arg-Lys-AOMK was demonstrated as a potent, neutral pH inhibitor of cathepsin B. These findings demonstrate that the distinct pH-dependent cleavage properties of cathepsin B can provide the basis for development of a neutral pH inhibitor with more than 100-fold greater potency at pH 7.2 compared to pH 4.6. The novel Z-Arg-Lys-AOMK inhibitor may allow future studies to probe the role of pathogenic neutral pH cathepsin B functions that participate in brain disorders and human diseases.

### Methods and Materials

Enzymes, peptides, and reagents. Recombinant human cathepsin B and cathepsin proteases were obtained from R&D Systems (Minneapolis, MN) or Abcam (Cambridge, MA) consisting of cathepsin B (R&D #953-CY-010), cathepsin L (R&D #952-CY-010), cathepsin V (R&D #1080-CY-010), cathepsin S (R&D #1183-CY-010), cathepsin K (Abcam #ab157067), cathepsin C (R&D #1071-CY-010), and cathepsin H (R&D #75116-CY-010). The design and synthesis of the 228 14-mer peptides used for MSP-MS assays have been described previously (54, 84). MSP-MS assays utilized low-bind 600 μL microtubes (Corning, Reynosa, MX), dithiothreitol (DTT) (Promega #V351. Madison, WI), urea (Teknova #U2222, Hollister, CA), HPLC-grade water (Fisher Chemical #W6-4), citric acid monohydrate (Merck #1.00244.0500, Burlington, MA), sodium phosphate dibasic anhydrous (EMD #SX-072305, Burlington, MA), sodium acetate (Fisher Scientific #BP-333-500, Fair Lawn, NJ), EDTA (Calbiochem #324503, Burlington, MA), sodium chloride (Fisher Chemical #S271-1, Pittsburgh, PA), acetonitrile (Fisher Chemical #A955-4, Pittsburgh, PA), formic acid (FA) (Fisher Chemical #A117-50, Pittsburgh, PA), trifluoroacetic acid (TFA) (Fisher Chemical #A116-50, Pittsburgh, PA), C18 LTS Tips (Rainin #PT-LC18-960, Oakland, CA), C18 for SPE stage-tips (3M company #2215-C18, Maplewood, MN), and BEH C18 packing material (Waters Corporation #186004661, Milford, MA). Fluorogenic peptide substrates were obtained from Bachem, Torrance; CA which consisted of Abz-Gly-Ile-Val-Arg-Ala-Lys(Dnp)-OH (SEQ ID NO: 1) (#4049308), Z-Arg-Arg-AMC (#4004789), Arg-AMC (#1-1050), Gly-Arg-AMC (#4002196). Z-Phe-Arg-AMC was purchased from Anaspec, Fremont, CA to (#AS-24096). Z-Lys-Lys-AMC, Z-Lys-Arg-AMC, Z-Arg-Lys-AMC, z-Glu-Lys-AMC and z-Glu-Arg-AMC were custom synthesized by Genscript (Piscataway, NJ). MCA-Arg-Pro-Pro-Gly-Phe-Ser-Ala-Phe-Lys(Dnp)OH (SEQ ID NO: 2) was from CPC Scientific, San Jose, CA (#AMYD-111A). E64c was from Selleckchem (Houston, TX); CA-074 and CA-074Me were from SigmaMillipore (Burlington, MA). Cell culture media components MEMalpha, F-12K, and FBS were from ThermoFisher (Watham, MA), and F-12K was from ATCC (Manassas, VA). The DC protein kit was from Biorad (Hercules, CA).

Cathepsin B activity and stability. Recombinant human pro-cathepsin B was activated to mature cathepsin B by incubation at 37° C. for 30 minutes in activation buffer (20 mM Na-acetate pH 5.5, 1 mM EDTA, 5 mM DTT, 100 mM NaCl). To examine enzyme activity and stability, cathepsin B was pre-incubated at pH 7.2 or pH 4.6 at room temperature (RT) (27° C.) and 37° C. for times of 0.5, 1, 2, 3, and 4 hours. Cathepsin B activity was then assayed at RT for 30 min in 50 mM citrate phosphate at pH 7.2 or pH 4.6, 40 mM Z-Phe-Arg-AMC substrate, 1 mM EDTA, 100 mM NaCl, 5 mM DTT, and 0.01% Tween20 with incubation at RT for 30 mM. Cleavage of Z-Phe-Arg-AMC to generate fluorescent AMC was monitored at excitation 360 nm and emission 460 nm. Assay conditions were conducted in triplicate and the mean±SD values were calculated.

Cathepsin B cleavage site analysis by multiplex substrate profiling by mass spectrometry (MSP-MS). Cathepsin B activity (activated) was titrated with E64c to calculate the concentration of active cathepsin B. For MSP-MS assays, cathepsin B (0.1 ng/μL, activated) was incubated with a peptide library of 228 14-mer peptides, each at 0.5 μM peptide, in buffer consisting of 50 mM citrate phosphate of pH 7.2 or pH 4.6, 1 mM EDTA, 100 mM NaCl and 4 mM DTT (total volume of 46 μL). After incubation for 15 and 60 mM at 25° C., 20 μL aliquots were removed and combined with 80 μL of 8 M urea. An inactivated cathepsin B control consisted of cathepsin B in assay buffer combined with 8 M urea for 60 minutes at 25° C. for denaturation, followed by addition of the peptide library. After incubation, samples were acidified by addition of 40 μL of 2% FA, desalted using C18 LTS Tips (Rainin), evaporated to dryness in a vacuum centrifuge, and stored at −70° C. Samples were resuspended in 20 μL of 0.1% FA (solvent A) and 1 μL was used for LC-MS/MS analysis. All MSP-MS conditions were conducted in quadruplicate assays.

MSP-MS assay samples were then subjected to LC-MS/MS performed on a Q-Exactive Mass Spectrometer (Thermo) equipped with an Ultimate 3000 HPLC (Thermo Fisher). Peptides were separated by reverse phase chromatography on a C18 column (1.7 μm bead size, 75 μm×20 cm, 65° C.) at a flow rate of 400 nL/min using solvent A (0.1% FA in water) and solvent B (0.1% FA in acetonitrile). LC separation was performed using a 50-minute linear gradient of 5% to 30% solvent B followed by a 15-15 minute linear gradient of 30% to 75% solvent B. Survey scans were recorded over a 200-2000 m/z range (70,000 resolutions at 200 m/z, AGC target 1×10⁶, 75 ms maximum). MS/MS was performed in data-dependent acquisition mode with HCD fragmentation (30 normalized collision energy) on the 10 most intense precursor ions (17,500 resolutions at 200 m/z, AGC target 5×10⁴, 120 ms maximum, dynamic exclusion 15 s).

Peak integration and peptide data analysis were performed using PEAKS (v 8.5) software (Bioinformatics Solutions Inc.). MS 2 data were searched against the 228-member tetradecapeptide library sequences and a decoy search was conducted with sequences in reverse order. A precursor tolerance of 20 ppm and 0.01 Da for MS 2 fragments was defined. No protease digestion was specified, Data were filtered to 1% peptide false discovery rates with the target-decoy strategy. Peptide intensities were quantified, and data was normalized by Loess-G algorithm (normalyzer.immunoprot.Ith.se/) and filtered by 0.5 peptide quality. Outliers from replicates were removed by Dixon's Q testing (91) when there were at least 3 replicate values found out of the 4 replicates for each condition for every peptide. Missing and zero values are imputed with random normally distributed numbers in the range of the average of smallest 5% of the data±SD. An ANOVA test was performed for peptide data found in the three conditions of control, 15 mM incubation, and 60 mm incubation; those with p<0.05 were considered for further analysis. Cleaved peptide products were defined as those with intensity scores of 8-fold or more above the quenched inactive cathepsin B, assessed using the ratio of log₂ (Cat.B/inactivated enzyme) for each peptide product. Ratios were evaluated for p<0.05 by 2-tailed homoscedastic t-test (data not shown).

Analyses of MSP-MS data for cleavage preferences of cathepsin B by iceLogo. Evaluation of the frequencies of amino acids adjacent to the cleavage sites was conducted using the iceLogo software (92). IceLogo analyses utilized (a) the ‘positive dataset’ consisted of the P2 to P2′ amino acids that surround the cleavage sites between the 12^{th} and 13^{th} amino acid of the 14-mer peptides and the (b) ‘negative dataset’ consisted of the P2 to P2′ amino acids for the 228 cleavage sites of the peptide library between the 12^{th} and 13^{th} amino acid. Analyses involved Z-scores calculated by the equation X−μ/σ, where X is the frequency of the amino acid in the experimental data set, μ is the frequency of a particular amino acid at a specific position in the reference set (control ‘0’ time), and is the standard deviation. Z-scores were utilized to generate iceLogo illustrations of the relative frequencies of amino acid residues at each of the P2 to P2′ positions of the cleaved peptides where heights of the single letter amino acids represent ‘percent difference’, defined as the difference in frequency for an amino acid appearing in the positive dataset relative to the negative dataset. Positive differences are shown above the midline, and negative differences are represented below the midline. Residues below the line shown in gray are those that were absent in the positive dataset.

Synthesis of Z-Arg-Lys-AOMK and Z-Glu-Lys-AOMK inhibitors. Inhibitor synthesis was achieved in three steps via production of Fmoc-Lys(Boc)-AOMK, semicarbazide aminomethyl polystyrene resin, 5, and Z-Arg-Lys-AOMK and Z-Glu-Lys-AOMK (data not shown).

For Fmoc-Lys (Boc)-AOMK synthesis, N-methylmorpholine (1.06 g, 10.5 mmol) and isobutyl chloroformate (1.434 g, 10.5 mmol) were added dropwise to a stirred solution of amino acid 1 (4.68 g, 10.0 mmol) in 100 mL dry tetrahydrofuran (THF) in a 200 mL flame polished round bottom flask at −10° C. After 15 min, ethereal diazomethane was generated and distilled from Diazald (6.43 g, 30.0 mmol) in accordance to procedures outlined in the Aldrich Technical Bulletin a1180 into stirred solution over the course of 30 min (AL-180) (Sigma Aldrich Technical Bulletin a1180 for Diazald and Diazomethane Generators). After distillation, the reaction was warmed to 25° C. and continued to stir for 1 hour. Glacial acetic acid was added dropwise after being chilled to quench excess diazomethane, and 33% HBr in acetic acid added dropwise until a red tint persisted for more than 5 min. The solvent was removed in vacuo, re-dissolved in ethyl acetate and subsequently washed with water, sat. aq. NaHCO₃ twice, sat. aq. NaCl, and dried over MgSO₄. A flame-dried 20 mL scintillation vial charged with anhydrous potassium fluoride (5 g, 100 mmol) and 2,6-dimethylbenzoic acid (7.509 g, 50 mmol) in 10 mL anhydrous dimethylformamide (DMF) was sonicated for 5 min. 2, dissolved in a minimal amount of anhydrous DMF, was added dropwise to stirred solution of carboxylic acid and base. After 30 min the solution was diluted with 250 mL ethyl acetate, washed twice with 200 mL sat. aq. NaCl, briefly with 1 M NaOH, sat. aq. NaHCO₃, sat. aq. NaCl, and dried over MgSO₄. The crude oil was purified by flash chromatography using 3:1 hexane:ethyl acetate to yield 3 in 81% yield.

For production of semicarbazide aminomethyl polystyrene resin, 5, a flame-dried 500 mL round bottom flask charged with a magnetic stir bar, aminomethyl polystyrene resin (25 g, 28.75 mmol), N,N′-carbonyldiimidazole (46.62 g, 287.5 mmol) in 250 mL anhydrous dichloromethane (DCM) was stirred under positive argon pressure for 3 h to generate 4 (data not shown). The resin was washed once with anhydrous DCM, once with anhydrous DMF, transferred into a new flame dried vessel and resuspended in 250 mL of anhydrous DMF. To this stirred solution, anhydrous hydrazine (55.29 g, 54.15 mL, 1725 mmol) was added gradually over 5 min. The reaction was stirred at room temperature for 1 h. The resin was filtered, washed 5 times with DCM, 5 times with MeOH, dried thoroughly in vacuo, and stored at 4° C.

A flame-dried 100 mL round bottom charged with amino acid 3 (1.09 g, 2.0 mmol) and 5 (1.00 g, 1.15 mmol/g) was dried in vacuo for 6 h and suspended in 20 mL anhydrous THF. This stirred solution was heated at 70° C. for 18 h to generate preloaded resin. Fmoc-Lys(Boc)-AOMK:SCR 6. The excess amino acid derivative was recovered and the resin washed twice each with DMF, DCM and MeOH, dried thoroughly and stored at −20° C.

For synthesis of Z-Arg-Lys-AOMK and Z-Glu-Lys-AOMK, preloaded resin 6 was presolvated in DCM for 30 min before two 15 min treatments of 5% diethylamine in DMF (1 mL/100 mg). Fmoc-glycine, HCTU, and N,N-diisopropylethylamine at a 3:3:10 ratio with respect to the loading of the resin was used to couple Z-Arg-OH or Z-Glu-OH. The resin was washed with DCM and MeOH 3× before being dried in vacuo. Cleavage was performed using 1 mL TFA:water:triisopropylsilane at a 95:2.5:2.5 ratio per 100 mg resin for 1 hour. The resin was washed with another aliquot of cleavage cocktail and the combined cleavage solutions were concentrated before precipitation with cold diethyl ether. The pellet was dried under a stream of argon and dissolved in a minimal volume of DMSO before purification by preparatory reverse phase HPLC (19×150 mm Xbridge C18, CH₃CMH₂O/0.1% TFA, 25:75 to 70:30 over 13 min:20 mL/min) and lyophilization.

Cathepsin B activity assayed by fluorogenic peptide substrates. Proteolytic assays of cathepsin B were conducted with Z-Arg-Lys-AMC and Z-Gly-Lys-AMC substrates in 50 mM citrate phosphate at pH 7.2 or pH 4.6, 1 mM EDTA, 100 mM NaCl, and 5 mM DTT. Assays with Z-Phe-Arg-AMC substrate were conducted under identical conditions and included 0.01% Tween20. Assays were performed in 384 well plates at 25° C. in a total volume of 30 μL. Fluorescence was quantified by a Biotek Synergy HTX microplate plate reader with excitation 360 nm, emission 460 nm, gain 50, top optics and read height 1 mm. Proteolytic activity is reported as relative fluorescent units per sec (RFU/sec), and was calculated using the highest slope recorded for 10 consecutive readings. Fluorescent readings were taken in 46 sec intervals and therefore activity is calculated over a total of 460 sec. To ensure that initial velocity is assessed, only readings within the first 30 minutes of the reaction were analyzed. RFU/sec readings were converted to enzyme specific activity of pmol/min/μg using the conversion factor of 1227 RFU per μM AMC.

For Michaelis-Menten kinetic characterization, assays contained 20 μL of substrate (Z-Phe-Arg-AMC, at different concentrations) and 10 μL of 0.125 ng/uL cathepsin B for a final enzyme concentration of 0.0417 ng/μL. The final concentrations of substrates were 225 μM to 2.6 μM with DMSO concentration of 4.5% v/v.

For the substrates Z-Arg-Lys-AMC, Z-Glu-Lys-AMC, and Z-Phe-Arg-AMC, k_{cat}/Kₘ values were calculated using the equation v₀=Vₘₐₓ*[S]/(Kₘ+[S]) where v₀ is the activity at a corresponding substrate concentration [S] and Vₘₐₓ is the maximum enzyme velocity at saturated [S] concentration. Vₘₐₓ=k_{cat}[E]T where [E]T is the total enzyme concentration Kₘ is the x-axis value where y=Vₘₐₓ/2 and Vₘₐₓ is the maximum rate at saturating substrate concentrations. At low [S], k_{cat}/Kₘ was calculated from the slope of the plot of v₀/[E] vs [S] concentration (linear portion of plot). All data was plotted, calculated, and analyzed using GraphPad Prism9 software.

To generate the pH profile of cathepsin B activity with the substrates Z-Arg-Lys-AMC, Z-Glu-Lys-AMC, and Z-Phe-Arg-AMC, 60 μM of each substrate and 0.04 ng/ul of cathepsin B were assayed in citrate phosphate buffers ranging from pH 2.2 to 7.4 in increments of 0.4 pH units, including pH 7.2. For assay buffers ranging from pH 7.4 to 9.0, 50 mM Tris-HCl was used instead of 50 mM citrate phosphate, with inclusion of pH 7.2.

Inhibitor kinetic characterization using fluorogenic assays for cathepsin B activity. Kinetic analyses of IC₅₀, K₁, k_{obs}, and k_{inact}/K₁ values for Z-Arg-Lys-AOMK and Z-Glu-Lys-AOMK inhibition of cathepsin B were conducted by fluorogenic proteolytic assays consisting of 40 μM Z-Phe-Arg-AMC, 40 mM citrate phosphate at pH 7.2 or pH 4.6, 1 mM EDTA, 100 mM NaCl, 5 mM DTT, and 0.01% Tween 20; assays were performed at room temperature (22-27° C.) in quadruplicate. Inhibitor and substrate were combined in the reaction well, and the assay was started upon addition of cathepsin B (0.04 ng/μL). The inhibitor concentration ranged from 5.5 μM to 1.1 nM (1.5-fold serial dilution). A vehicle control assay contained 2% DMSO instead of inhibitor. Enzyme velocity (RFU/sec) was measured during a 30 mM incubation period as relative fluorescent units per sec (RFU/s), calculated using the highest slope recorded for 10 consecutive readings taken at 46 sec intervals (thus, activity is calculated over a total of 460 sec). Prism software was used to analyze enzyme activity data in kinetic studies. IC₅₀ values were calculated (without preincubation of inhibitor and enzyme) as the concentration of inhibitor that reduced cathepsin B activity by 50%.

For determination of K₁ and k_{inact}/K₁ kinetic inhibition constants, k_{obs} constants were determined by plots of cathepsin B activity in time courses with different inhibitor concentrations by curve fitting slope data of RFU versus time into Y=Y₀*e^{(-k}_{obs}*^{X)}, where Y₀ is the activity for the control with no inhibitor condition, Y is the activity in the presence of inhibitor, X is time. K₁ and k_{inact} values were calculated from curve fitting the k_{obs} values into the equation k_{obs}=k_{inact}*[I]/(K₁+[I]), where [I] is inhibitor concentration, and K₁ is the x-axis inhibitor concentration where y=k_{inact}/2 and k_{inact} is the maximum rate of inactivation at saturating inhibitor concentrations (93, 94). These kinetic analyses are for irreversible inhibitors, Z-Arg-Lys-AOMK and Z-Glu-Lys-AOMK of this study, that utilize Ki values, rather than Ki values used for reversible inhibitors (93).

Irreversible mechanism of inhibitors. The irreversible or reversible mechanism of cathepsin B inhibition was assessed for Z-Arg-Lys-AOMK and Z-Glu-Lys-AOMK inhibitors. At pH 7.2, cathepsin B was incubated with 190 nM Z-Arg-Lys-AOMK and 4.99 μM Z-Glu-Lys-AOMK for 15 min in pH 7.2 assay buffer and the assay was performed as described above for pH 4.6. Proteolytic activity was monitored for 2 hr. At pH 4.6, cathepsin B (activated, 3.7 ng/μL) was incubated with 4.24 μM Z-Arg-Lys-AOMK and 3.48 μM Z-Glu-Lys-AOMK for 15 min in 40 mM citrate phosphate pH 4.6, 1 mM EDTA, 100 mM NaCl, and 5 mM DTT; a vehicle control contained 2.5% DMSO. Each reaction was then diluted 100-fold in assay buffer such that the final assay composition was 0.04 ng/μL cathepsin B, 40 μM Z-Phe-Arg-AMC, and an inhibitor concentration of 1/10^{th} the IC₅₀ value.

Inhibitor inhibition of peptide cleavages characterized by MSP-MS. MSP-MS assays in the presence of inhibitors were performed as outlined above except cathepsin B (0.1 ng/μL) was pre-incubated with Z-Arg-Lys-AOMK (64 nM) or a vehicle control (0.5% DMSO) for 30 mM at 25° C. prior to incubation with the peptide library for 60 min in 40 mM citrate phosphate at pH 7.2 or pH 4.6, 1 mM EDTA, 100 mM NaCl and 5 mM DTT buffer. Assays were conducted in quadruplicate and immediately stored at −70° C. following quenching with 8M urea.

Specificity of inhibitors for other cysteine cathepsin proteases. The effects of Z-Arg-Lys-AOMK and Z-Glu-Lys-AOMK on cathepsin V, L, K, S, X, H and C activities were assessed. IC₅₀ values were calculated at pH 4.6 and pH 7.2 conditions, consisting of 40 mM citrate phosphate, 1 mM EDTA, 100 mM NaCl, and 5 mM DTT. The inhibitor concentrations ranged from 16.38 μM to 256 nM with 2-fold serial dilutions. When activity (RFU/s) in the presence of 16.38 μM inhibitor was reduced by <50% compared to DMSO control, the IC₅₀ value was indicated as >16 μM. Cathepsin V (0.04 ng/μL), cathepsin L (0.03 ng/μL), cathepsin K (0.10 ng/μL), cathepsin S (0.20 ng/uL) were assayed with 40 μM Z-Phe-Arg-AMC. Cathepsin X (0.20 ng/uL), cathepsin C (0.51 ng/uL) and cathepsin H (0.1 ng/μL) were assayed with 40 μM of MCA-Arg-Pro-Pro-Gly-Phe-Ser-Ala-Phe-Lys(Dnp)OH (SEQ ID NO: 2), Gly-Arg-AMC, and Arg-AMC, respectively. Activation of pro-cathepsin H to cathepsin H was conducted by incubation of cathepsin H (4.4 ng/μL) with cathepsin L (1.1 ng/μL) at RT for 2 hrs in activation buffer (20 mM citrate phosphate pH 6.0, 100 mM NaCl, and 5 mM DTT). Cathepsin C (13.78 ng/μL) was activated by incubation with cathepsin L (3.4 ng/μL) at RT for one hr in activation buffer (20 mM citrate phosphate, pH 6.0, 100 mM NaCl, 5 mM DTT). It was confirmed that cathepsin L did not cleave the cathepsin C and cathepsin H substrates Gly-Arg-AMC and Arg-AMC, respectively. For all assays containing peptide-AMC substrates the fluorescent microplate reader settings were the same as outlined above for cathepsin B. For the cathepsin X assay, the plate reader was set to excitation 320 nm, emission 400 nm, gain 105, top optics and read height 1 mm. To convert RFU/s to picomol/min, 10 μM to 0.005 μM (2-fold serial dilution) of MCA-Arg-Pro-Pro-Gly-Phe-Ser-Ala-Phe-Lys(Dnp)OH (SEQ ID NO: 2) was fully hydrolyzed with excess cathepsin X and a standard curve was generated using the total fluorescence values calculated at each concentration.

MOE modeling of Z-Arg-Lvs-AOMK binding interactions with cathepsin B. The Molecular Operating Environment (MOE) modeling software (65, 66) was used to model Z-R-K-AOMK binding to cathepsin B using the crystal structure of cathepsin B (PDB 1QDQ) (61), co-crystal template with the inhibitor CA-074 as default binding ligand. The builder function of MOE was used to examine binding poses that considered polar contacts and hydrogen bonds between ligand and the active site pocket of 1QDQ at pH 7.2, Docking simulations were performed with energy-minimized structures to assess ligand flexibility and poses using the MOE docking feature.

Cathepsin B in human neuroblastoma cells treated with inhibitors. Neuroblastoma cells (human SHSY5Y) were grown in media consisting of 50% MEMalpha, 50% F12-K, 10% heat-inactivated FBS at 37° C. in an atmosphere of 95% air and 5% CO₂. Firstly, cell homogenates were prepared by collection of cells in 0.32 M sucrose and freeze-thawing. Secondly, cells were incubated with Z-Arg-Lys-AOMK or CA-074Me for 6 hours at 37° C., and washed 3 times in phosphate-buffered saline and homogenates were prepared in 0.32 M sucrose with freeze-thawing. CA-074Me is a methyl ester form of the active CA-074 selective inhibitor of cathepsin B (70); CA-074Me penetrates the cell and is converted by intracellular esterases to CA-074.

Cathepsin B activity in the homogenates were monitored with Z-Arg-Arg-AMC substrate (60 μM) with buffer conditions of 40 mM citrate-phosphate, pH 5.5, 5 mM DTT, 1 mM EDTA, 100 mM NaCl, and 1.2% DMSO, followed by incubation at 37° C. for 30 minutes and reading of AMC fluorescence. CA-074-senstive activity was monitored to indicate cathepsin B activity, since CA-074 is a selective inhibitor of this enzyme (70). Protein content was measured in homogenates with the DC protein assay kit. Cathepsin B specific activity was calculated as nmol AMC/μg/min, and reported as the mean±SD (with analyses for statistical significance, p<0.05 by student's t-test).

Further details and data not shown may be found at pubs.acs.org/doi/10.1021/acschembio.1c001387fig=fig1&ref=pdf& published as ACS Chem Biol. 2021 Sep. 17; 16(9):1628-1643. doi: 10.1021/acschembio.1c00138. Epub 2021 Aug. 20.

### REFERENCES

1. De Duve, C., Wattiaux, R. (1966) Functions of lysosomes, Annu Rev Physiol. 28, 435-92.
2. Lawrence, RE., Zoncu, R. (2019) The lysosome as a cellular centre for signaling, metabolism and quality control, Nat Cell Biol. 21, 133-142.
3. Xu, H., Ren, D. (2015) Lysosomal physiology, Annu Rev Physiol. 77, 57-80.
4. Barrett, A.J., Rawlings, N.D., and Woessner, J.F. (2004). Handbook of Proteolytic Enzymes, 2nd edition, Elsevier Academic Press, Amsterdam.
5. Turk, V., Stoka, V., Vasiljeva, O., Renko, M., Sun, T., Turk, B., Turk, D. (2012) Cysteine cathepsins: from structure, function and regulation to new frontiers, Biochim Biophys Acta. 2012 1824, 68-88.
6. Mindell, JA. Lysosomal acidification mechanisms, Annu Rev Physiol. 74, 69-86,
7. Ishida, Y., Nayak, S., Mindell, J.A., Grabe, M. (2013) A model of lysosomal pH regulation, J Gen Physiol. 141, 705-20.
8. Ohkuma, S., Poole, B. (1978) Fluorescence probe measurement of the intralysosomal pH in living cells and the perturbation of pH by various agents, Proc Natl Acad Sci USA 75, 3327-31.
9. Bright, G.R., Fisher, G.W., Rogowska, J., Taylor, D.L.)1987) Fluorescence ratio imaging microscopy: temporal and spatial measurements of cytoplasmic pH, J Cell Biol. 104, 1019-33.
10. Madshus, I.H. (1988) Regulation of intracellular pH in eukaryotic cells, Biochem J. 250, 1-8.
11. Swietach, P., Tiffert, T., Mauritz, J.M., Seear, R., Esposito, A., Kaminski, C.F., Lew. V.L., Vaughan-Jones, R.D. (2010) Hydrogen ion dynamics in human red blood cells, J Physiol. 588, 4995-5014.
12. Hook, V., Yoon, M., Mosier, C., Ito, G., Podvin, S., Head, B.P., Rissman, R., O'Donoghue, A.J., Hook, G. (2020) Cathepsin B in neurodegeneration of Alzheimer's disease, traumatic brain injury, and related brain disorders. Biochim Biophys Acta Proteins Proteom. 1868, 140428.
13. Nakanishi, H. (2020) Microglial cathepsin B as a key driver of inflammatory brain diseases and brain aging, Neural Regen Res.15, 25-29.
14. Yang, A.J., Chandswangbhuvana, D., Margol, L., Glabe, C.G. (1998) Loss of endosomal/lysosomal membrane impermeability is an early event in amyloid Abetal-42 pathogenesis, J Neurosci Res. 52, 691-8.
15. Ditaranto, K., Tekirian, T.L., Yang, A.J. (2001) Lysosomal membrane damage in soluble Abeta-mediated cell death in Alzheimer's disease, Neurobiol Dis. 8, 19-31.
16. De Kimpe, L., van Haastert, E.S., Kaminari, A., Zwart, R., Rutjes, H., Hoozemans, J.J., Scheper, W. (2013) Intracellular accumulation of aggregated pyroglutamate amyloid beta: convergence of aging and Aβ pathology at the lysosome, Age 35, 673-87.
17. Umeda, T., Tomiyama, T., Sakama, N., Tanaka, S., Lambert, M.P., Klein, W.L., Mori, H. (2011) Intraneuronal amyloid β oligomers cause cell death via endoplasmic reticulum stress, endosomal/lysosomal leakage, and mitochondrial dysfunction in vivo, J Neurosci Res. 89, 1031-42.
18. Lafrenaye, A.D., McGinn, M.J., Povlishock, J.T. (2012) Increased intracranial pressure after diffuse traumatic brain injury exacerbates neuronal somatic membrane poration but not axonal injury: evidence for primary intracranial pressure-induced neuronal perturbation, J Cereb Blood Flow Metab. 32, 1919-32.
19. Luo, C.L., Chen, X.P., Li, L.L., Li, Q.Q., Li, B.X., Xue, A.M., Xu, H.F., Dai, D.K., Shen, Y.W., Tao, L.Y., Zhao, Z.Q. (2013) Poloxamer 188 attenuates in vitro traumatic brain injury-induced mitochondrial and lysosomal membrane permeabilization damage in cultured primary neurons, J Neurotrauma 30, 597-607.
20. Windelbom, J.A., Lipton, P. (2008) Lysosomal release of cathepsins causes ischemic damage in the rat hippocampal slice and depends on NMDA-mediated calcium influx, arachidonic acid metabolism, and free radical production, J Neurochem. 106, 56-69.
21. Kilinc, M., Gürsoy-Ozdemir, Y., Gürer, G., Erdener, S.E., Erdemli, E., Can, A., Dalkara, T. (2010) Lysosomal rupture, necroapoptotic interactions and potential crosstalk between cysteine proteases in neurons shortly after focal ischemia, Neurobiol Dis. 40, 293-302.
22. Dong, H., Qin, Y., Huang, Y., Ji, D., Wu, F. (2019) Poloxamer 188 rescues MPTP-induced lysosomal membrane integrity impairment in cellular and mouse models of Parkinson's disease, Neurochem Int. 126, 178-186.
23. Freeman, D., Cedillos, R., Choyke, S., Lukic, Z., McGuire, K., Marvin, S., Burrage, A.M., Sudholt, S., Rana, A., O'Connor, C., Wiethoff, C,M., Campbell, E.M. (2013) Alpha-synuclein induces lysosomal rupture and cathepsin dependent reactive oxygen species following endocytosis, PLoS One 8, e62143.
24. Amritraj, A., Peake, K., Kodam, A., Salio, C., Merighi, A., Vance, J.E., Kar, S. (2009) Increased activity and altered subcellular distribution of lysosomal enzymes determine neuronal vulnerability in Niemann-Pick type Cl-deficient mice, Am J Pathol 175, 2540-56.
25. Chung, C., Puthanveetil, P., Ory, D.S., Lieberman, A.P. (2016) Genetic and pharmacological evidence implicates cathepsins in Niemann-Pick C cerebellar degeneration, Hum Mol Genet 25, 1434-46.
26. Fujisawa, A., Kambe, N., Saito, M., Nishikomori, R., Tanizaki, H., Kanazawa, N., Adachi, S., Heike, T., Sagara, J., Suda, T., Nakahata, T., Miyachi, Y. (2007) Disease-associated mutations in CIAS1 induce cathepsin B-dependent rapid cell death of human THP-1 monocytic cells, Blood 109, 2903-11.
27. Fujisawa, A., Kambe, N., Saito, M., Nishikomori, R., Tanizaki, H., Kanazawa, N., Adachi, S., Heike, T., Sagara, J., Suda, T., Nakahata, T., Miyachi, Y. (2007) Disease-associated mutations in CIAS1 induce cathepsin B-dependent rapid cell death of human THP-1 monocytic cells, Blood 109, 2903-11.
28. Rajamäki, K., Lappalainen, J., Oörni, K., Välimäki, E., Matikainen, S., Kovanen, P.T., Eklund, K.K. (2010) Cholesterol crystals activate the NLRP3 inflammasome in human macrophages: a novel link between cholesterol metabolism and inflammation, PLoS One 5, e11765.
29. Gonzalez, E.A., Martins, G.R., Tavares, A.M.V., Viegas, M., Poletto, E., Giugliani, R., Matte, U., Balch, G. (2018) Cathepsin B inhibition attenuates cardiovascular pathology in mucopolysaccharidosis I mice, Life Sci 196, 102-109.
30. Saluja, A., Dudeja, V., Dawra, R., Sah, R.P. (2019) Early Intra-Acinar Events in Pathogenesis of Pancreatitis, Gastroenterology 156, 1979-1993.
31. Amaral, E.P., Riteau, N., Moayeri, M., Maier, N., Mayer-Barber, K.D., Pereira, R.M., Lage, S.L., Kubler, A., Bishai, W.R., D'Império-Lima, M.R., Sher, A., Andrade, B.B. (2018) Lysosomal Cathepsin Release Is Required for NLRP3-Inflammasome Activation by Mycobacterium tuberculosis in Infected Macrophages, Front Immunol 9, 1427.
32. Morchang, A., Panaampon, J., Suttitheptumrong, A., Yasamut, U., Noisakran, S., Yenchitsomanus, P.T., Limjindapom, T. (2013) Role of cathepsin B in dengue virus-mediated apoptosis, Biochem Biophys Res Commun 438, 20-5.
33. de Castro, M.A., Bunt, G., Wouters, F.S. (2016) Cathepsin B launches an apoptotic exit effort upon cell death-associated disruption of lysosomes, Cell Death Discov 2, 16012.
34. Droga-Mazovec, G., Bojic, L., Petelin, A., Ivanova, S., Romih, R., Repnik, U., Salvesen, G.S., Stoka, V., Turk, V., Turk, B. (2008) Cysteine cathepsins trigger caspase-dependent cell death through cleavage of bid and antiapoptotic Bcl-2 to homologues, J Biol Chem 283, 19140-50.
35. Kavčič, N., Pegan, K., Turk, B. (2017) Lysosomes in programmed cell death pathways: from initiators to amplifiers. Biol Chem 398, 289-301.
36. Wei, M.C., Lindsten, T., Mootha, V.K., Weiler, S., Gross, A., Ashiya, M. Thompson, C.B., Korsmeyer, S.J. (2000) tBID, a membrane-targeted death ligand, oligomerizes BAK to release cytochrome c, Genes Dev 14, 2060-71.
37. Campden, R.I., Zhang, Y. (2019) The role of lysosomal cysteine cathepsins in NLRP3 inflammasome activation, Arch Biochem Biophys 670, 32-42.
38. Bai, H., Yang, B., Yu, W., Xiao, Y., Yu, D., Zhang, Q. (2018) Cathepsin B links oxidative stress to the activation of NLRP3 inflammasome, Exp Cell Res 362, 180-187.
39. Lian, D., Lai, J., Wu, Y., Wang, L., Chen, Y., Zhang, Y., Boini, K.M., Huang, Y., Chen, Y. (2018) Cathepsin B-mediated NLRP3 inflammasome formation and activation in angiotensin II -Induced hypertensive mice: role of macrophage digestion dysfunction, Cell Physiol Biochem 50, 1585-1600.
40. Amaral, E.P., Riteau, N., Moayeri, M., Maier, N., Mayer-Barber, K.D., Pereira, R.M., Lage, S.L., Kubler, A., Bishai, W.R., D'Imperio-Lima, M.R., Sher, A., Andrade, B.B. (2018) Lysosomal cathepsin release is required for NLRP3-inflammasome activation by mycobacterium tuberculosis in infected macrophages. Front Immunol 9, 1427.
41. Kindy, M.S., Yu, J., Zhu, H., El-Amouri, S.S., Hook, V., Hook, G.R. (2012) Deletion of the cathepsin B gene improves memory deficits in a transgenic Alzheimer's disease mouse model expressing AβPP containing the wild-type β-secretase site sequence, J Alzheimers Dis 29, 827-40.
42. Hook, G.R., Yu, J., Sipes, N., Pierschbacher, M.D., Hook, V., Kindy, M.S. (2014) The cysteine protease cathepsin B is a key drug target and cysteine protease inhibitors are potential therapeutics for traumatic brain injury, J Neurotrauma 31, 515-29.
43. Buck, M.R., Karustis, D.G., Day, N.A., Honn, KV., Sloane, B.F. (1992) Degradation of extracellular-matrix proteins by human cathepsin B from normal and tumour tissues, Biochem J 282, 273-8.
44. Cavallo-Medved, D., Dosescu, J., Linebaugh, B.E., Sameni, M., Rudy, D., Sloane, B.F. (2003) Mutant K-ras regulates cathepsin B localization on the surface of human colorectal carcinoma cells, Neoplasia 5, 507-19.
45. Victor, B.C., Anbalagan, A., Mohamed, M.M., Sloane, B.F., Cavallo-Medved ,D. (2011) Inhibition of cathepsin B activity attenuates extracellular matrix degradation and inflammatory breast cancer invasion. Breast Cancer Res 13, R115.
46. Aggarwal, N., Sloane, B.F. (2014) Cathepsin B: multiple roles in cancer, Proteomics Clin Appl 8, 427-37.
47. Bian, B., Mongrain, S., Cagnol, S., Langlois, M.J., Boulanger, J., Bernatchez, G., Carrier, J.C., Boudreau, F., Rivard, N. (20106) Cathepsin B promotes colorectal tumorigenesis, cell invasion, and metastasis, Mol Carcinog 55, 671-87.
48. Mort, J.S., Recklies, A.D., Poole, A.R. (1984) Extracellular presence of the lysosomal proteinase cathepsin B in rheumatoid synovium and its activity at neutral pH, Arthritis Rheum 27, 509-15.
49. Jordans, S., Jenko-Kokalj, S., Kühl, N.M., Tedelind, S., Sendt, W., Brömme, D., Turk, D., Brix, K. (2009) Monitoring compartment-specific substrate cleavage by cathepsins B, K, L, and S at physiological pH and redox conditions, BMC Biochem 20, 23.
50. Tedelind, S., Poliakova, K., Valeta, A., Hunegnaw, R., Yemanaberhan, E.L., Heldin, N.E., Kurebayashi. J., Weber, E., Kopitar-Jerala, N., Turk, B., Bogyo, M., Brix, K. (2010) Nuclear cysteine cathepsin variants in thyroid carcinoma cells, Biol Chem 391, 923-35.
51. Hämälistö, S., Stahl, J.L., Favaro, E., Yang, Q., Liu, B Christoffersen, L., Loos, B., Boldú, C., Joyce, J.A., Reinheckel, T., Barisic, M., Jäättelä, M. (2020) Spatially and temporally defined lysosomal leakage facilitates mitotic chromosome segregation, Nat Commun 11, 229.
52. Almeida, P.C., Nantes, I.L., Chagas, J.R., Rizzi, C.C., Faljoni-Alario, A., Carmona, E., Juliano, L., Nader, H.B., Tersariol, I.L. (2001) Cathepsin B activity regulation. Heparin-like glycosaminogylcans protect human cathepsin B from alkaline pH-induced inactivation, J Biol Chem 276, 44-51.
53. Pratt, M.R., Sekedat, M.D., Chiang, K.P., Muir, T.W. (2009) Direct measurement of cathepsin B activity in the cytosol of apoptotic cells by an activity-based probe, Chem Biol 16, 1001-12.
54. O'Donoghue, A.J., Eroy-Reveles, A.A., Knudsen, G.M., Ingram, J., Zhou, M., Stamekov, J.B., Greninger, A.L., Hostetter, D.R., Qu, G., Maltby, D.A., Anderson, M.O., Derisi, J.L., McKerrow, J.H., Burlingame, A.L., Craik, C.S. (2012) Global identification of peptidase specificity by multiplex substrate profiling, Nat Methods 9, 1095-100.
55. Lapek, J.D. Jr, Jiang, Z., Wozniak, J.M., Arutyunova, E., Wang, S.C., Lemieux, M.J, Gonzalez, D.J., O'Donoghue, A.J. (2019) Quantitative Multiplex Substrate Profiling of Peptidases by Mass Spectrometry, Mol Cell Proteomics 8, 968-981..
56. Ivry, S.L., Knudsen, G.M., Caiazza, F., Sharib, J.M., Jaradeh, K., Ravalin, M., O'Donoghue, A.J., Kirkwood, K.S., Craik, C.S. (2019) The lysosomal aminopeptidase tripeptidyl peptidase 1 displays increased activity in malignant pancreatic cysts, Biol Chem 400, 1629-1638.
57. Xu, J.H., Jiang, Z., Solania, A., Chatterjee, S., Suzuki, B., Lietz, C.B., Hook, V.Y.H., O'Donoghue, A.J., Wolan, D.W. (2018) A Commensal Dipeptidyl Aminopeptidase with Specificity for N-Terminal Glycine Degrades Human-Produced Antimicrobial Peptides in Vitro. ACS Chem Biol 13, 2513-2521.
58. Leontovyč, A., Ulrychová, L., O'Donoghue, A.J., Vondrášek, J., Marešová, L., Hubálek, M., Fajtová, P., Chanová, M., Jiang, Z., Craik, C.S., Caffrey, C.R., Mareš, M., Dvoŕák, J., Horn, M. (2018) SmSP2: A serine protease secreted by the blood fluke pathogen Schistosoma mansoni with anti-hemostatic properties, PLoS Negl Trop Dis 12, 00006446.
59. Li, H., Goh, B.N., Teh, W.K., Jiang, Z., Goh, J.P.Z., Goh, A., Wu, G., Hoon, S.S., Raida, M., Camattari, A., Yang, L., O'Donoghue, A.J., Dawson, T.L. Jr. (2018) Skin Commensal Malassezia globosa Secreted Protease Attenuates Staphylococcus aureus Biofilm Formation, J Invest Dermatol 138, 1137-1145.
60. Beekman, C., Jiang, Z., Suzuki. B.M., Palmer, J.M., Lindner, D.L., O′Donoghue, A.J., Knudsen, G.M., Bennett, R.J. (2018) Characterization of PdCP1, a serine carboxypeptidase from Pseudogymnoascus destructans, the causal agent of White-nose Syndrome, Biol Chem 399, 1375-1388.
61. Yamamoto, A., Tomoo, K., Hara, T., Murata, M., Kitamura, K., Ishida, T. (2000) Substrate specificity of bovine cathepsin B and its inhibition by CA074, based on crystal structure refinement of the complex, J Biochem 127, 635-43.
62. Thornberry, N.A., Peterson, E.P., Zhao, J.J., Howard, A.D., Griffin, P.R., Chapman, K.T. (1994) Inactivation of interleukin-1 beta converting enzyme by peptide (acyloxy)methyl ketones, Biochemistry 33, 3934-40.
63. Albrow, V.E., Ponder, E.L., Fasci, D., Békés, M., Deu, E., Salvesen, G.S., Bogyo, M. (2011) Development of small molecule inhibitors and probes of human SUMO deconjugating proteases, Chem Biol 18, 722-32.
64. Shan, L. Carbobenzoxy-capped Phe-Lys(Cy5)-acyloxymethyl ketone. 2010 May 9 [updated 2010 May 25]. In: Molecular Imaging and Contrast Agent Database (MICAD) [Internet]. Bethesda (MD): National Center for Biotechnology Information (US); 2004-2013.
65. Vilar, S., Cozza, G., Moro, S. (2008) Medicinal chemistry and the molecular operating environment (MOE): application of QSAR and molecular docking to drug discovery. Curr Top Med Chem 8, 1555-72.
66. Roy, U., Luck, L.A. (2007) Molecular modeling of estrogen receptor using molecular operating environment, Biochem Mol Biol Educ 35, 238-43.
67. Schechter, I. (2005) Mapping of the active site of proteases in the 1960s and rational design of inhibitors/drugs in the 1990s, Curr Protein Pept Sci 6, 501-12.
68. Hasnain, S., Hirama. T., Huber, C.P., Mason, P., Mort, J.S. (1993) Characterization of cathepsin B specificity by site-directed mutagenesis. Importance of Glu245 in the S2-P2 specificity for arginine and its role in transition state stabilization, J Biol Chem 268, 235-40.
69. Musil ; D., Zucic, D., Turk, D., Engh, R A., Mayr, I., Huber, R., Popovic, T., Turk, V., Towatari, T., Katunuma, N., et al. The refined 2.15 Å X-ray crystal structure of human liver cathepsin B: the structural basis for its specificity, EMBO J 10, 2321-30.
70. Murata, M., Miyashita, S., Yokoo, C., Tamai, M., Hanada, K., Hatayama, K., Towatari, T., Nikawa, T., Katunuma, N. (1991) Novel epoxysuccinyl peptides. Selective inhibitors of cathepsin B, in vitro, FEBS Lett 280, 307-10.
71. Buttle, D.J., Murata, M., Knight, C.G., Barrett, A.J. (1992) CA074 methyl ester: a proinhibitor for intracellular cathepsin B. Arch Biochem Biophys 299, 377-80.
72. Choe, Y., Leonetti, F., Greenbaum, D.C., Lecaille, F., Bogyo, M., Bromme, D., Ellman, J.A., Craik, C.S. (2006) Substrate profiling of cysteine proteases using a combinatorial peptide library identifies functionally unique specificities, J Biol Chem 281, 12824-32.
73. Poreba, M., Groborz, K., Vizovisek, M., Maruggi, M., Turk, D., Turk, B., Powis, G., Drag, M., Salvesen, G.S. (2019) Fluorescent probes towards selective cathepsin B detection and visualization in cancer cells and patient samples, Chem Sci 10, 8461-8477.
74. Turk, B., Dolenc, I., Zerovnik, E., Turk, D., Gubensek, F., Turk, V. (1994) Human cathepsin B is a metastable enzyme stabilized by specific ionic interactions associated with the active site, Biochemistry 33, 14800-6.
75. Song, J., Xu, P., Xiang, H., Su, Z., Storer, A.C., Ni, F. (2000) The active-site residue Cys-29 is responsible for the neutral-pH inactivation and the refolding barrier of human cathepsin B, FEBS Lett 475, 157-62.
76. Vidak, E., JavorSek, U., Vizovišek, M., Turk, B. (2019) Cysteine Cathepsins and their Extracellular Roles: Shaping the Microenvironment, Cells 8, 264.
77. Spizz, G., Blackshear, P.J. (1997) Identification and characterization of cathepsin B as the cellular MARCKS cleaving enzyme. J Biol Chem 272, 23833-42.
78. Maciewicz, R.A., Wotton, S.F., Etherington, D.J., Duance, V.C. (1990) Susceptibility of the cartilage collagens types II, IX and XI to degradation by the cysteine proteinases, cathepsins B and L, FEBS Lett 269, 189-93.
79. Taha, T.A., Kitatani, K., Bielawski, J., Cho, W., Hannun, Y.A., Obeid, L.M. (2005) Tumor necrosis factor induces the loss of sphingosine kinase-1 by a cathepsin B-dependent mechanism, J Biol Chem 280, 17196-202.
80. Illy, C., Quraishi, O., Wang, J., Purisima, E., Vernet, T., Mort, J.S. (1997) Role of the occluding loop in cathepsin B activity, J Biol Chem 72, 1197-202.
81. Nägler. D.K., Storer, A.C., Portaro, F,C., Carmona, E., Juliano, L., Ménard, R. (1997) Major increase in endopeptidase activity of human cathepsin B upon removal of occluding loop contacts, Biochemistry 36, 12608-15.
82. Melo, R.L., Pozzo, R.C., Alves, L.C., Perissutti, E., Caliendo, G., Santagada, V., Juliano, L., Juliano, M.A. (2001) Synthesis and hydrolysis by cathepsin B of fluorogenic substrates with the general structure benzoyl-X-ARG-MCA containing non-natural basic amino acids at position X, Biochim Biophys Acta 1547, 82-94.
83. Linebaugh, B.E., Sameni, M., Day, N.A., Sloane, B.F., Keppler, D. (1999) Exocytosis of active cathepsin B enzyme activity at pH 7.0, inhibition and molecular mass, Eur J Biochem 264, 100-9.
84. Boutté, A.M., Hook, V., Thangavelu, B., Sarkis, G.A., Abbatiello, B.N., Hook, G., Jacobsen, J.S., Robertson, C.S., Gilsdorf, J., Yang, Z., Wang, K.K.W., Shear, D.A. (2020) Penetrating Traumatic Brain Injury Triggers Dysregulation of Cathepsin B Protein Levels Independent of Cysteine Protease Activity in Brain and Cerebral Spinal Fluid, J Neurotrauma 37, 1574-1586.
85. Hoegen, T., Tremel, N., Klein, M., Angele, B., Wagner, H., Kirschning, C., Pfister, H.W., Fontana, A., Hammerschmidt, S., Koedel, U. (2011) The NLRP3 inflammasome contributes to brain injury in pneumococcal meningitis and is activated through ATP-dependent lysosomal cathepsin B release, J Immunol 187, 5440-51.
86. Yamashima T. (2016) Can ‘calpain-cathepsin hypothesis’ explain Alzheimer neuronal death? Ageing Res Rev 32, 169-179.
87. Yamashima T. (2012) Hsp70.1 and related lysosomal factors for necrotic neuronal death. J Neurochem 120, 477-94.
88. Zhang, X., Lin, Y., Gillies, R.J. (2010) Tumor pH and its measurement, J Nucl Med 51, 1167-70.
89. Chen, L.Q., Pagel, M.D. (2015) Evaluating pH in the Extracellular Tumor Microenvironment Using CEST MRI and Other Imaging Methods, Adv Radiol 2015, 206405.
90. O'Donoghue, A.J., Knudsen, G.M., Beekman, C., Perry, J A., Johnson, A.D., DeRisi, J.L., Craik, C.S., Bennett, R.J. (2015) Destructin-1 is a collagen-degrading endopeptidase secreted by Pseudogymnoascus destructans, the causative agent of white-nose syndrome, Proc Natl Acad Sci USA 112, 7478-83.
91. Rorabacher, D.B. (1991) Statistical treatment for rejection of deviant values: critical values of Dixon's Q parameter and related subrange ratios at the 95% confidence level, Anal Chem 63, 139-146.
92. Colaert, N., Helsens, K., Martens, L., Vandekerckhove, J., Gevaert, K. (2009) Improved visualization of protein consensus sequences by iceLogo, Nat Methods 6, 786-787.
93. Strelow, J.M. (2017) A Perspective on the Kinetics of Covalent and Irreversible Inhibition, SLAS Discov 22, 3-20.
94. Tonge, P.J. (2019) Quantifying the Interactions between Biomolecules: Guidelines for Assay Design and Data Analysis, ACS Infect Dis 5, 796-808.
95. Acta Pharm Sin B. 2015 Nov;5(6):506-19. PMID: 26713267; doi: 10.1016/j.apsb.2015.08.001.
96. J Med Chem. 2020 Nov 12;63(21):12725-12747. PMID: 33054210; doi: 10.1021/acs.jmedchem.0c01063.
97. Ada Biomater . 2014 Apr;10(4):1671-82. doi: 10.1016/j.actbio.2013.08.013.
98. tcichemicals.com/US/en/c/10247.
99. Zenón-Meléndez CN, Carrasquillo Carrión K, Cantres Rosario Y, Roche Lima A, Meléndez LM. Inhibition of Cathepsin B and SAPC Secreted by HIV-Infected Macrophages Reverses Common and Unique Apoptosis Pathways. J Proteome Res. 2022 Feb 4;21(2):301-312.
100. Proc Natl Acad Sci USA, 1986 Apr;83(8):2483-7 doi: 10.1073/pnas.83.8.2483.
101. Ann Rheum Dis., 1995 Apr;54(4):289-97. doi: 10.1136/ard.54.4.289.

1. A peptidic inhibitor of cathepsin B comprising an amino-terminally capped di-peptide Arg-Lys carboxy-terminally conjugated to a cysteine protease inhibitor, wherein the peptidic inhibitor of cathepsin B inhibits cathepsin B activity at neutral pH more effectively than at lower pH.

2. The peptidic inhibitor of cathepsin B of claim 1, wherein the cysteine protease inhibitor is acyloxymethyl ketone (AOMK).

3. The peptidic inhibitor of cathepsin B of claim 1, wherein the amino-terminal cap is a benzyloxycarbonyl (Z).

4. The peptidic inhibitor of cathepsin B of claim 1, comprising Z-Arg-Lys-AOMK (Z-R-K-AOMK).

5. A pharmaceutical composition comprising the peptidic inhibitor of cathepsin B of claim 1, and a pharmaceutically acceptable excipient.

6. A method of inhibiting neutral pH cathepsin B activity, comprising administering to a mammalian subject in need an effective amount of a peptidic inhibitor of cathepsin B comprising an amino-terminally capped di-peptide Arg-Lys carboxy-terminally conjugated to a cysteine protease inhibitor, wherein the peptidic inhibitor of cathepsin B inhibits cathepsin B activity at neutral pH more effectively than at lower pH.

7. The method of claim 6, wherein the cysteine protease inhibitor is acyloxymethyl ketone (AOMK).

8. The method of claim 6, wherein the amino-terminal cap is a benzyloxycarbonyl (Z).

9. The method of claim 6, wherein the peptidic inhibitor of cathepsin B comprises Z-Arg-Lys-AOMK (Z-R-K-AOMK).

10. The method of claim 6, wherein the neutral pH cathepsin B activity is due to lysosomal leakage of cathepsin B to neutral cytosol, nucleus or extracellular locations.

11. A method for treatment of a disease or disorder characterized by lysosomal leakage of cathepsin B, comprising administering to a mammalian subject in need an effective amount of a pharmaceutical composition comprising a peptidic inhibitor of cathepsin B comprising an amino-terminally capped di-peptide Arg-Lys carboxy-terminally conjugated to a cysteine protease inhibitor, wherein the peptidic inhibitor of cathepsin B inhibits cathepsin B activity at neutral pH more effectively than at lower pH.

12. The method of claim 11, wherein the cysteine protease inhibitor is acyloxymethyl ketone (AOMK).

13. The method of claim 11, wherein the amino-terminal cap is a benzyloxycarbonyl (Z).

14. The method of claim 11, wherein the peptidic inhibitor of cathepsin B comprises Z-Arg-Lys-AOMK (Z-R-K-AOMK).

15. The method of claim 11, wherein the disease or condition is neurological.

16. The method of claim 11, wherein the disease or condition is inflammatory, infectious or metabolic.

17. The method of claim 11, wherein the disease is cancer.

18. The method of claim 11, wherein the condition is characterized by cytosolic cathepsin B initiated apoptotic cell death or activation of inflammatory IL-1beta production.

19. The method of claim 11, wherein the disease or condition occurs at a neutral pH site in the subject.

20. The method of claim 19, wherein the neutral pH site in the subject is within a nucleus, plasma membrane, or extracellular space.
